(19) United States
(12) Patent Application Publication
(10) Pub. No.: US 2024/0083982 A1
(43) Pub. Date: Mar. 14, 2024

(54) THERAPEUTIC NEUTRALIZING ANTIBODIES FOR SARS-COV-2

(71) Applicants: California Institute of Technology, Pasadena, CA (US); Protabit LLC, Pasadena, CA (US)

(72) Inventors: Barry D. Olafson, Pasadena, CA (US); Stephen L. Mayo, Pasadena, CA (US); Pamela J. Bjorkman, Altadena, CA (US); Jost G. Vielmetter, Pasadena, CA (US); Justin W. Chartron, Pasadena, CA (US); Paul M. Chang, Pasadena, CA (US); Stephanie C. Contreras, Folsom, CA (US); Jingzhou Wang, Pasadena, CA (US); Aiden J. Aceves, Pasadena, CA (US); Anthony P. West, Jr., Pasadena, CA (US); Christopher O. Barnes, Pasadena, CA (US); Jennifer R. Keeffe, Los Angeles, CA (US); Claudia A. Jette, Pasadena, CA (US)

(21) Appl. No.: 18/356,810

(22) Filed: Jul. 21, 2023

Related U.S. Application Data
(60) Provisional application No. 63/391,444, filed on Jul. 22, 2022.

(51) Int. Cl. C07K 16/10; A61K 39/42; A61K 45/06; A61P 31/14

(52) U.S. Cl. CPC C07K 16/1003; A61K 39/42; A61K 45/06; A61P 31/14; C07K 2317/55; C07K 2317/622

(57) ABSTRACT

Disclosed herein include antibodies or fragments thereof having specificity to a sarbecovirus spike protein. Also provided are compositions, methods, and kits for using said antibodies or fragments thereof for preventing or treating, for example a coronavirus infection.

### RELATED APPLICATIONS

This application claims the benefit under 35 U.S.C. § 119(e) of U.S. Provisional Patent Application Ser. No. 63/391,444, filed Jul. 22, 2022. The content of these related application is incorporated herein by reference in its entirety for all purposes.

### STATEMENT REGARDING FEDERALLY SPONSORED R&D

This invention was made with government support under Grant No(s). IIP2027586 awarded by the National Science Foundation. The government has certain rights in the invention.

### REFERENCE TO SEQUENCE LISTING

The present application is being filed along with a Sequence Listing in electronic format. The Sequence Listing is provided as a file entitled 30KJ-302432-US_SeqList, created Jul. 2, 2023, which is 195,884 bytes in size. The information in the electronic format of the Sequence Listing is incorporated herein by reference in its entirety.

### BACKGROUND

### Field

The present disclosure relates generally to the field of biopharmaceuticals.

### Description of the Related Art

Spillover of animal SARS-like betacoronaviruses (sarbecoviruses) resulted in two human health emergencies in the early 21^{st} century: the SARS-CoV epidemic in the early 2000s and the COVID-19 pandemic caused by SARS-CoV-2 beginning in late 2019. Large coronavirus reservoirs in bats are predictive of future cross-species transmission, necessitating vaccines and therapies that can protect against emerging coronaviruses. In addition, SARS-CoV-2 variants have emerged throughout the COVID-19 pandemic, with the Alpha, Beta, Delta, Gamma, and Omicron lineages designated as variants of concern (VOCs) due to apparent increased transmissibility and/or resistance to neutralizing antibodies elicited by infection or vaccination. In the case of Omicron VOCs, a large number of substitutions in the SARS-CoV-2 spike protein receptor-binding domain (RBD), the major target of neutralizing antibodies and detectable cross-variant neutralization, results in reduced efficacies of vaccines and therapeutic monoclonal antibodies (mAbs).

Comparison of the variability of RBDs across sarbecoviruses and within SARS-CoV-2 VOCs and variants of interest (VOIs) suggest that vaccines and mAbs targeting the more conserved neutralizing antibody epitopes (class 4 and class 1/4) can protect against present and future SARS-CoV-2 VOCs and prevent future sarbecovirus spillover events from causing another epidemic or pandemic. By contrast, antibodies targeting the less conserved class 1 and class 2 RBD epitopes that directly overlap with the binding footprint for human ACE2, the SARS-CoV-2 host receptor, recognize a portion of the RBD that exhibits sequence variability between sarbecoviruses, which is also where VOC and VOI substitutions accumulate. Class 3 RBD epitopes are more conserved than class 1 and class 2 epitopes but exhibit some variation across sarbecoviruses, suggesting the potential for continued variability amongst SARS-CoV-2 VOCs. (WO2023102476).

There is a need for antibody or antibody fragment capable of broadly and effectively neutralizing sarbecoviruses and be useful, for example, for treating or preventing SARS-Cov-2 infections, as well as infections due to emerging coronaviruses.

### SUMMARY

Disclosed herein include antibodies or fragments thereof having specificity to one or more sarbecoviruses. The antibody or a fragment thereof can, for example, comprise: (a) a heavy chain variable region (i.e. VH) CDR-H1 (i.e. heavy chain complementarity-determining region 1 or heavy chain CDR1) comprising an amino acid sequence selected from SEQ ID NOs: 44-75 or a variant thereof having a single substitution, deletion or insertion from any one of SEQ ID NOs: 44-75; (b) a VH CDR-H2 comprising an amino acid sequence selected from SEQ ID NOs: 76-107 or a variant thereof having a single substitution, deletion or insertion from any one of SEQ ID NOs: 76-107; (c) a VH CDR-H3 comprising an amino acid sequence selected from SEQ ID NOs: 108-139 or a variant thereof having a single substitution, deletion or insertion from any one of SEQ ID NOs: 108-139; (d) a light chain variable region (i.e. VL) CDR-L1 comprising an amino acid sequence selected from SEQ ID NOs: 140-150 or a variant thereof having a single substitution, deletion or insertion from any one of SEQ ID NOs: 140-150; (e) a VL CDR-L2 comprising an amino acid sequence selected from SEQ ID NOs: 151-161 or a variant thereof having a single substitution, deletion or insertion from any one of SEQ ID NOs: 151-161; and (f) a VL CDR-L3 comprising an amino acid sequence selected from SEQ ID NOs: 162-172 or a variant thereof having a single substitution, deletion or insertion from any one of SEQ ID NOs: 162-172.

The antibody or fragment thereof can comprise: (a) a VH CDR-H1 comprising an amino acid sequence selected from SEQ ID NOs: 44-75; (b) a VH CDR-H2 comprising an amino acid sequence selected from SEQ ID NOs: 76-107; (c) a VH CDR-H3 comprising an amino acid sequence selected from SEQ ID NOs: 108-139; (d) a VL CDR-L1 comprising an amino acid sequence selected from SEQ ID NOs: 140-150; (e) a VL CDR-L2 comprising an amino acid sequence selected from SEQ ID NOs: 151-161; and (f) a VL CDR-L3 comprising an amino acid sequence selected from SEQ ID NOs: SEQ ID NOs: 162-172.

A single chain Fab fragment (scFab) or a scFab fragment thereof, of the antibody or the fragment thereof is disclosed herein, wherein the scFab or scFab fragment thereof comprises a heavy chain of the antibody fragment and a light chain of the antibody fragment, wherein a scFab linker connects a C-terminal of the heavy chain to an N-terminal the light chain as described.

A single chain variable region antibody (scFv) or a scFv fragment thereof, of the antibody or the fragment thereof is disclosed herein, wherein the single chain variable region antibody (scFv) or a scFv fragment thereof comprises a heavy chain variable region of the antibody fragment and a light chain variable region of the antibody fragment, wherein a scFab linker connects a C-terminal of the heavy chain variable region and an N-terminal the light chain variable region as described herein.

Disclosed herein include scFv or a scFv fragment thereof of having an amino acid sequence selected from SEQ ID NOs: 186-196, or a variant thereof having a single substitution, deletion, or insertion from SEQ ID NOs: 186-196.

A variant of the amino acid sequences of the antibody or fragment thereof can have at least 90% sequence identity to the amino acid sequence. Thus, a variant is disclosed for any one of the amino acid sequence of SEQ ID NOs: 1-196, each variant of which can have at least 90% sequence identity to the corresponding amino acid sequence of SEQ ID NOs: 1-196.

In some embodiments, the antibody or fragment thereof comprises an Fc domain. In some embodiments, the antibody or fragment thereof is a scFv, a single-domain antibody, an immunoglobulin molecule, a monoclonal antibody, a chimeric antibody, a CDR-grafted antibody, a humanized antibody, a Fab fragment, a Fab′ fragment, a F(ab′)₂ fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof.

In some embodiments, the antibody or fragment thereof specifically binds to one or more sarbecoviruses. In some embodiments, the antibody or fragment thereof binds to one or more sarbecoviruses with an EC50 of less than 10 μg/mL, less than 1 μg/mL, less than 0.1 μg/mL, less than 0.01 μg/mL, less than 0.001 μg/mL, or less than 0.0001 μg/mL as assessed by SARS2 pseudovirus assay, an optofluidic system and/or an ELISA assay.

In some embodiments, the antibody or fragment thereof binds to one or more sarbecoviruses with an EC50 of less than 10 μg/mL, less than 1 μg/mL, less than 0.1 μg/mL, or less than 0.01 μg/mL less than 0.001 μg/mL, or less than 0.0001 μg/mL as assessed by SARS2 pseudovirus assay, an optofluidic system and/or an ELISA assay. In some embodiments, the antibody or fragment thereof binds to one or more sarbecoviruses with an EC50 of about 0.0001 μg/mL to about 10 μg/mL, about 0.001 μg/mL to about 1μg/mL, about 0.001 μg/mL to about 0.1 μg/mL, or about 0.001 μg/mL to about 0.01 μg/mL as assessed by SARS2 pseudovirus assay, an optofluidic system and/or an ELISA assay. In some embodiments, the antibody or fragment thereof binds to one or more sarbecoviruses with an EC50 of about 0.0001 μg/mL to about 10 μg/mL, about 0.001 μg/mL to about 1 μg/mL, about 0.001 μg/mL to about 0.1 μg/mL, or about 0.001 μg/mL to about 0.01 μg/mL as assessed by SARS2 pseudovirus assay, an optofluidic system and/or an ELISA assay.

In some embodiments, the antibody or fragment thereof inhibits infectivity of one or more sarbecoviruses with an IC50 less than 10 μg/mL, less than 1 μg/mL, less than 0.1 μg/mL or less than 0.01 μg/mL. In some embodiments, IC50 is measured by a pseudovirus neutralization assay. In some embodiments, the antibody or fragment thereof inhibits infectivity of one or more sarbecoviruses with an IC50 of about 0.0001 μg/mL to about 10 μg/mL, about 0.001 μg/mL to about 1 μg/mL, about 0.001 μg/mL to about 0.1 μg/mL, or about 0.001 μg/mL to about 0.01 μg/mL.

In some embodiments, the antibody or fragment thereof inhibits infectivity of one or more sarbecoviruses. In some embodiments, the antibody or fragment thereof inhibits infectivity of at least one, at least two, or all of the two or more viruses with an IC50 less than 10 μg/mL, less than 1 μg/mL, less than 0.1 μg/mL, or less than 0.01 μg/mL. In some embodiments, the antibody or fragment thereof inhibits infectivity of at least one, at least two, or all of the two or more viruses with an IC50 of about 0.0001 μg/mL to about 10 μg/mL, about 0.001 μg/mL to about 1 μg/mL, about 0.001 μg/mL to about 0.1 μg/mL, or about 0.001 μg/mL to about 0.01 μg/mL. In some embodiments, the antibody or fragment thereof inhibits infectivity of at least one, at least two, or all of the two or more viruses with an IC50 of about 0.005 μg/mL to about 9μg/mL, e.g., an IC50 of about 0.001 μg/mL to about 0.06 μg/mL, an IC50 of about 0.02 μg/mL to about 6 μg/mL, or an IC50 of about 0.002 μg/mL to about 2 μg/mL. In some embodiments, the pseudovirus neutralization assay comprises target cells expressing hACE2 receptor protein.

The sarbecovirus can be SARS-CoV-2 or a variant thereof, SARS-CoV or a variant thereof, B.1.1.7 (20I/501Y.V1) or a variant thereof, B.1.351 (20H/501Y.V2) or a variant thereof, P.1 (20J/501Y.V3) or a variant thereof, WIV1 or a variant thereof, SHC014 or a variant thereof, BtKY72 or a variant thereof. In some embodiments, the antibody can bind specifically to Khosta2/SARS-CoV Chimera RBD or variants thereof, or LyRa3/SARS-CoV RBD Chimera or variants thereof. In some embodiments, the SARS-CoV-2 variants comprise Wuhan (WA1 D614G), Beta, Delta, Omicron BA.1, Omicron BA.2, Omicron BA.4, and Omicron BA.5.

Disclosed herein include compositions comprising any of the antibodies or fragments thereof provided herein and a pharmaceutically acceptable carrier. Also disclosed herein are polynucleotides encoding one or more of the antibodies or fragments thereof provided herein. There are provided isolated cells comprising any of the polynucleotides provided herein. Disclosed herein include compositions comprising any of the polynucleotides and/or an isolated cells provided herein.

Disclosed herein include methods of treating or preventing a coronavirus infection in a patient in need thereof, e.g., administering to the patient an effective amount of any of the antibodies or fragments thereof, polynucleotides, isolated cells, and compositions provided herein, or a combination thereof. In some embodiments, the coronavirus is a coronavirus in the genus of Alpha-coronavirus, Beta-coronavirus, or both. In some embodiments, the coronavirus is a coronavirus of the subgenus Sarbecovirus. In some embodiments, the coronavirus is SARS-CoV-2 or a variant thereof, B.1.1.7 (20I/501Y.V1) or a variant thereof, B.1.351 (20H/501Y.V2) or a variant thereof, P.1 (20J/501Y.V3) or a variant thereof, RsSTT200 or a variant thereof, Pang17 or a variant thereof, RaTG13 or a variant thereof, SARS-CoV or a variant thereof, WIV1 or a variant thereof, SHC014 or a variant thereof, LyRa3 or a variant thereof, C028 or a variant thereof, Rs4081 or a variant thereof, RmYN02 or a variant thereof, Rf1 or a variant thereof, Yun11 or a variant thereof, BM4831 or a variant thereof, BtKY72 or a variant thereof, or Khosta2 or a variant thereof. In some embodiments, the coronavirus comprises a SARS-CoV-2 variant of concern, variant of interest, or both. In some embodiments, the SARS-CoV-2 variant of concern comprises SARS-CoV-2 Beta and variants thereof, B.1.1.7 (20I/501Y.V1), B.1.351 (20H/501Y.V2), P.1 (20J/501Y.V3), SARS-CoV-2 Delta and variants thereof, or SARS-CoV-2 Omicron and variants thereof.

The method can comprise administering to the patient a second therapeutic agent (e.g., an anti-viral compound, an immunosuppressant, an antibody, or any combination thereof. In some embodiments, the second therapeutic agent comprises remdesivir, molnupiravir, tocilizumab, favipiravir, merimepodib, artesunate, favipiravir, ribavirin, EIDD-2801, niclosamide, nitazoxanide, oseltamivir, AT-527, paxlovid, regdanvimab, ramdicivir, baricitinib, imatinib, casirivimab, imdevimab, bemcentinib, bamlanivimab, etesevimab, sotrovimab, leronlimab, bebtelovimab, cilgavimab, IMU-838, oseltamivir, or dexamethasone).

### BRIEF DESCRIPTION OF THE DRAWINGS

FIG. 1 depicts a table showing characteristics of SARS-CoV-2 variants of concern—worldwide, September 2020-January 2021. (Summer E. Galloway et al. “Emergence of SARS-CoV-2 B.1.1.7 Lineage—United States, Dec. 29, 2020-Jan. 12, 2021, US Department of Health and Human Services/Centers for Disease Control and Prevention, Morbidity and Mortality Weekly Report, Jan. 22, 2021/Vol. 70/No. 3).

FIG. 2 depicts a 96-well assay format of an in-house poly-L-Lys coated plate free of polybrene for the pseudovirus assays as described herein, for example SARS-CoV-2 501Y.V2 or SARS2-CoV-2 B1.1.7 as indicated in FIG. 1. Data was read robotically. Format: 12 point, 3-fold dilution.

FIG. 3 depicts SARS2 pseudovirus assay with antibody variants showing results of percentage neutralization vs. antibody concentrations for C002 V1 (a heavy chain amino acid sequence of SEQ ID NO: 174 and a light chain amino acid sequence of SEQ ID NO: 173) (solid circle, IC50 0.0035 μg/mL), C002 V2 (a heavy chain amino acid sequence of SEQ ID NO: 175 and a light chain amino acid sequence of SEQ ID NO: 173) (solid square, IC50 0.0004 μg/mL), C002 V3 (a heavy chain amino acid sequence of SEQ ID NO: 176 and a light chain amino acid sequence of SEQ ID NO: 173) (solid triangle, IC50 0.0024 μg/mL), C002 V4 (a heavy chain amino acid sequence of SEQ ID NO: 177 and a light chain amino acid sequence of SEQ ID NO: 173) (solid diamond, IC50 0.40 μg/mL) and C002 V5 (a heavy chain amino acid sequence of SEQ ID NO: 178 and a light chain amino acid sequence of SEQ ID NO: 173) (blank triangle, IC50 0.0033 μg/mL).

FIG. 4 depicts SARS2 pseudovirus assay with antibody variants showing results of percentage neutralization vs. antibody concentrations for C002 V2(a heavy chain amino acid sequence of SEQ ID NO: 175 and a light chain amino acid sequence of SEQ ID NO: 173) (solid circle, IC50 0.028 μg/mL), C002 V7 (a heavy chain amino acid sequence of SEQ ID NO: 180 and a light chain amino acid sequence of SEQ ID NO: 173) (solid square, IC50 0.13 μg/mL), C002 V9 (a heavy chain amino acid sequence of SEQ ID NO: 181 and a light chain amino acid sequence of SEQ ID NO: 173) (solid triangle, IC50 0.0036 μg/mL), C002 V10 (a heavy chain amino acid sequence of SEQ ID NO: 182 and a light chain amino acid sequence of SEQ ID NO: 173) (solid diamond, IC50 0.001 μg/mL) and C005 IgG (blank triangle, IC50 0.001 μg/mL).

FIG. 5 depict a table showing results in FIG. 3 and FIG. 4 and presenting a ratio of IC50 for each antibody variant to WT IgG for V1, V3, V4, V5, V6, V7 and V9 and a ratio of IC50 for WT IgG to V2 and V10.

FIG. 6 depict SARS2-CoV-2 D614G pseudovirus assay with antibody variants showing results of percentage neutralization vs. antibody concentrations for C002 V1 (a heavy chain amino acid sequence of SEQ ID NO: 174 and a light chain amino acid sequence of SEQ ID NO: 173) (lower solid square, IC50 6.5 vg/mL), C002 V2 (a heavy chain amino acid sequence of SEQ ID NO: 175 and a light chain amino acid sequence of SEQ ID NO: 173) (solid triangle, IC50 73 ng/mL), C002 V3 (a heavy chain amino acid sequence of SEQ ID NO: 176 and a light chain amino acid sequence of SEQ ID NO: 173) (solid diamond, IC50 1.8 ng/mL), C002 V4 (a heavy chain amino acid sequence of SEQ ID NO: 177 and a light chain amino acid sequence of SEQ ID NO: 173) (upper solid square, IC50 0.7 ng/mL) and C002 V5(a heavy chain amino acid sequence of SEQ ID NO: 178 and a light chain amino acid sequence of SEQ ID NO: 173) (solid circle, IC50 5.5 ng/mL), 1:6 virus dilution (new stock of virus).

FIG. 7 depicts SARS2-CoV-2 D614G pseudovirus assay with antibody variants showing results of percentage neutralization vs. antibody concentrations for C002 V6 (a heavy chain amino acid sequence of SEQ ID NO: 179 and a light chain amino acid sequence of SEQ ID NO: 173) (upper solid circle, IC50 1.4 ng/mL), C002 V7 (a heavy chain amino acid sequence of SEQ ID NO: 180 and a light chain amino acid sequence of SEQ ID NO: 173) (solid square, IC50 3.2 ng/mL), C002 V9 (a heavy chain amino acid sequence of SEQ ID NO: 181 and a light chain amino acid sequence of SEQ ID NO: 173) (solid triangle, IC50 10 ng/mL), C002 V10 (a heavy chain amino acid sequence of SEQ ID NO: 182 and a light chain amino acid sequence of SEQ ID NO: 173) (solid diamond, IC50 410 ng/mL) and C002 IgG (lower solid circle, IC50 7.2 ng/mL), 1:6 virus dilution (new stock of virus).

FIG. 8 depicts a table showing results in FIG. 6 and FIG. 7 and presenting a ratio of IC50 for each antibody variant to WT IgG for V2, V9 and V10 and a ratio of IC50 for WT IgG to V1, V3, V4, V5, V6, and V7. Included in the Table are IC50 for C101 IgG and C105 IgG. IC 50 for C002 IgG, C101 IgG and C105 IgG are reported in literature to be 8.9 ng/mL, 8.2 ng/mL, and 26 ng/mL respectively. The assay data were analyzed by a 5-parameter fit.

FIG. 9 depicts SARS2 pseudovirus assay with antibody variant showing results of percentage neutralization vs. antibody concentrations for C002 V4 (a heavy chain amino acid sequence of SEQ ID NO: 177 and a light chain amino acid sequence of SEQ ID NO: 173) (solid circle, assay of Dec. 11, 2020; and solid square, assay of Dec. 17, 2020).

FIG. 10 depicts SARS2 pseudovirus assay with antibody variant showing results of percentage neutralization vs. antibody concentrations for C002 V10 (a heavy chain amino acid sequence of SEQ ID NO: 182 and a light chain amino acid sequence of SEQ ID NO: 173) (solid circle, assay of Dec. 11, 2020; and solid square, assay of Dec. 17, 2020).

FIG. 11 depicts SARS2 pseudovirus assay with antibody C002 WT showing results of percentage neutralization vs. antibody concentrations for C002 WT (solid circle, assay of Dec. 11, 2020; and solid square, assay of Dec. 17, 2020).

FIG. 12 depicts a table showing duplicate results of SARS2 pseudovirus assay for c002 V1, V2, V3, V4, V5, V6, V7, V9 and V10 on two separate days and presenting IC50 in ng/mL and a ratio of IC50 for each antibody variant to WT IgG for c002 V1, V2, V3, V4, V5, V6, V7, V9 and V10.

FIG. 13 depicts SARS2 pseudovirus assay with antibody variant C002 V4 showing results of percentage neutralization vs. antibody concentrations for C002 V4 (solid circle) and C002 IgG (solid square).

FIG. 14 depicts a table showing triplicate results of SARS2 pseudovirus assay for c002 V4 and WT IgG on three separate days and presenting corresponding IC50 data in ng/mL.

FIG. 15 depicts SARS2-CoV-2 UK isolate (B.1.1.7) pseudovirus assay with antibody variants showing results of percentage neutralization vs. antibody concentrations for C002 V6 (solid triangle, IC50 1.0 ng/mL), C002 V7 (solid diamond, IC50 0.1 ng/mL), C002 V12 (a heavy chain amino acid sequence of SEQ ID NO: 184 and a light chain amino acid sequence of SEQ ID NO: 173) (solid circle, IC50 2.1 ng/mL), and C002 IgG (solid square, IC50 6.1 ng/mL), 1:6 virus dilution (new stock of virus).

FIG. 16 depicts a table showing triplicate results of SARS2-CoV-2 UK isolate (B.1.1.7) pseudovirus assay for c002 V4, V6, V7, V12, and WT IgG on different days and presenting IC50 in ng/mL for each antibody variant and WT IgG. As indicated in FIG. 1, the mutations in UK isolate include H69-70V deletion, Y144 deletion, N501Y, A570D, D614G, P681H, T7161, S982A and D1118H which correspond to positions in the c002 epitopes.

FIG. 17 depicts SARS2-CoV-2 UK isolate (B.1.1.7) and D614G (“WT”) pseudovirus assay with antibody variants showing results of percentage neutralization vs. antibody concentrations for C002 V7 (B.1.1.7) (solid triangle, IC50 0.3 ng/mL), C002 V7 (D614G) (solid circle, IC50 0.4 ng/mL), and C002 IgG (B.1.1.7) (solid diamond, IC50 2.6 ng/mL), C002 IgG (D614G) (solid square, IC50 11.2 ng/mL). As indicated in FIG. 1, the mutations in UK isolate include H69-70V deletion, Y144 deletion, N501Y, A570D, D614G, P681H, T716I, S982A and D1118H which correspond to positions in the c002 epitopes. IC 50 for C002 IgG against WT is reported in literature to be 8.9 ng/mL.

FIG. 18 depicts a table showing duplicate results of SARS2-CoV-2 UK isolate (B.1.1.7) and D614G (“WT”) pseudovirus assay for V7, and WT IgG on different days and presenting IC50 in ng/mL for c002 V7 antibody variant and WT IgG.

FIG. 19 depicts SARS2-CoV-2 UK isolate (B.1.1.7) pseudovirus assay with antibody variants showing results of percentage neutralization vs. antibody concentrations for C002 V6 (solid triangle, IC50 1.0 ng/mL), C002 V7 (solid diamond, IC50 0.1 ng/mL), and C002 V12 (solid circle, IC50 2.1 ng/mL), and C002 IgG (solid square, IC50 6.1 ng/mL). As indicated in FIG. 1, the mutations in UK isolate include H69-70V deletion, Y144 deletion, N501Y, A570D, D614G, P681H, T716I, S982A and D1118H which correspond to positions in the c002 epitopes. IC 50 for C002 IgG against WT is reported in literature to be 8.9 ng/mL.

FIG. 20 is a table showing results of SARS2-CoV-2 UK isolate (B.1.1.7) and D614G (“WT”) pseudovirus assay for V4, V6, V7, V12, and WT IgG on different days and presenting IC50 in ng/mL for c002 V4, V6, V7, V12, and antibody variants and c002 WT IgG, respectively.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include antibodies or fragments thereof having specificity to a coronavirus antigen. In some embodiments, the antibody or fragment thereof has specificity to a coronavirus spike protein, e.g., the RBD of the spike protein. The antibodies and fragments thereof disclosed herein can, for example, neutralize coronaviruses (e.g., SARS-CoV-2).

The antibody or fragment thereof can comprise: a VH CDR-H1 of SEQ ID NO: 44 or a variant thereof having a single substitution, deletion or insertion of SEQ ID NO: 44; a VH CDR-H2 of SEQ ID NO: 76 or a variant thereof having a single substitution, deletion or insertion of SEQ ID NO: 76; a VH CDR-H3 of SEQ ID NO: 108 or a variant thereof having a single substitution, deletion or insertion of SEQ ID NO: 108; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 45 or a variant thereof having a single substitution, deletion or insertion of SEQ ID NO: 45; a VH CDR-H2 of SEQ ID NO: 77 or a variant thereof having a single substitution, deletion or insertion of SEQ ID NO: 77; a VH CDR-H3 of SEQ ID NO: 109 or a variant thereof having a single substitution, deletion or insertion of SEQ ID NO: 109; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 46 or a variant thereof having a single substitution, deletion or insertion of SEQ ID NO: 46; a VH CDR-H2 of SEQ ID NO: 78 or a variant thereof having a single substitution, deletion or insertion of SEQ ID NO: 78; a VH CDR-H3 of SEQ ID NO: 110 or a variant thereof having a single substitution, deletion or insertion of SEQ ID NO: 110; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 47 or a variant thereof having a single substitution, deletion or insertion of SEQ ID NO: 47; a VH CDR-H2 of SEQ ID NO: 79 or a variant thereof having a single substitution, deletion or insertion of SEQ ID NO: 79; a VH CDR-H3 of SEQ ID NO: 111 or a variant thereof having a single substitution, deletion or insertion of SEQ ID NO: 111; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 48 or a variant thereof having a single substitution, deletion or insertion of SEQ ID NO: 48; a VH CDR-H2 of SEQ ID NO: 80 or a variant thereof having a single substitution, deletion or insertion of SEQ ID NO: 80; a VH CDR-H3 of SEQ ID NO: 112 or a variant thereof having a single substitution, deletion or insertion of SEQ ID NO: 112; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 49 or a variant thereof having a single substitution, deletion or insertion of SEQ ID NO: 49; a VH CDR-H2 of SEQ ID NO: 81 or a variant thereof having a single substitution, deletion or insertion of SEQ ID NO: 81; a VH CDR-H3 of SEQ ID NO: 113 or a variant thereof having a single substitution, deletion or insertion of SEQ ID NO: 113; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 50 or a variant thereof having a single substitution, deletion or insertion of SEQ ID NO: 50; a VH CDR-H2 of SEQ ID NO: 82 or a variant thereof having a single substitution, deletion or insertion of SEQ ID NO: 82; a VH CDR-H3 of SEQ ID NO: 114 or a variant thereof having a single substitution, deletion or insertion of SEQ ID NO: 114; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 51 or a variant thereof having a single substitution, deletion or insertion of SEQ ID NO: 51; a VH CDR-H2 of SEQ ID NO: 83 or a variant thereof having a single substitution, deletion or insertion of SEQ ID NO: 83; a VH CDR-H3 of SEQ ID NO: 115 or a variant thereof having a single substitution, deletion or insertion of SEQ ID NO: 115; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 52 or a variant thereof having a single substitution, deletion or insertion of SEQ ID NO: 52; a VH CDR-H2 of SEQ ID NO: 84 or a variant thereof having a single substitution, deletion or insertion of SEQ ID NO: 84; a VH CDR-H3 of SEQ ID NO: 116 or a variant thereof having a single substitution, deletion or insertion of SEQ ID NO: 116; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 53 or a variant thereof having a single substitution, deletion or insertion of SEQ ID NO: 53; a VH CDR-H2 of SEQ ID NO: 85 or a variant thereof having a single substitution, deletion or insertion of SEQ ID NO: 85; a VH CDR-H3 of SEQ ID NO: 117 or a variant thereof having a single substitution, deletion or insertion of SEQ ID NO: 117; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 54 or a variant thereof having a single substitution, deletion or insertion of SEQ ID NO: 54; a VH CDR-H2 of SEQ ID NO: 86 or a variant thereof having a single substitution, deletion or insertion of SEQ ID NO: 86; a VH CDR-H3 of SEQ ID NO: 118 or a variant thereof having a single substitution, deletion or insertion of SEQ ID NO: 118; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 55 or a variant thereof having a single substitution, deletion or insertion of SEQ ID NO: 55; a VH CDR-H2 of SEQ ID NO: 87 or a variant thereof having a single substitution, deletion or insertion of SEQ ID NO: 87; a VH CDR-H3 of SEQ ID NO: 119 or a variant thereof having a single substitution, deletion or insertion of SEQ ID NO: 119; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 56 or a variant thereof having a single substitution, deletion or insertion of SEQ ID NO: 56; a VH CDR-H2 of SEQ ID NO: 88 or a variant thereof having a single substitution, deletion or insertion of SEQ ID NO: 88; a VH CDR-H3 of SEQ ID NO: 120 or a variant thereof having a single substitution, deletion or insertion of SEQ ID NO: 120; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 57 or a variant thereof having a single substitution, deletion or insertion of SEQ ID NO: 57; a VH CDR-H2 of SEQ ID NO: 89 or a variant thereof having a single substitution, deletion or insertion of SEQ ID NO: 89; a VH CDR-H3 of SEQ ID NO: 121 or a variant thereof having a single substitution, deletion or insertion of SEQ ID NO: 121; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 58 or a variant thereof having a single substitution, deletion or insertion of SEQ ID NO: 58; a VH CDR-H2 of SEQ ID NO: 90 or a variant thereof having a single substitution, deletion or insertion of SEQ ID NO: 90; a VH CDR-H3 of SEQ ID NO: 122 or a variant thereof having a single substitution, deletion or insertion of SEQ ID NO: 122; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 59 or a variant thereof having a single substitution, deletion or insertion of SEQ ID NO: 59; a VH CDR-H2 of SEQ ID NO: 91 or a variant thereof having a single substitution, deletion or insertion of SEQ ID NO: 91; a VH CDR-H3 of SEQ ID NO: 123 or a variant thereof having a single substitution, deletion or insertion of SEQ ID NO: 123; a VL CDR-L1 of SEQ ID NO: 140 or a variant thereof having a single substitution, deletion or insertion of SEQ ID NO: 140; a VL CDR-L2 having the amino acid sequence of SEQ ID NO: 151 or a variant thereof having a single substitution, deletion or insertion of SEQ ID NO: 151; and a VL CDR-L3 of SEQ ID NO: 162 or a variant thereof having a single substitution, deletion or insertion of SEQ ID NO: 162.

The antibody or fragment thereof can, for example, comprise: a VH CDR-H1 of SEQ ID NO: 60 or a variant thereof having a single substitution, deletion or insertion of SEQ ID NO: 60; a VH CDR-H2 of SEQ ID NO: 92 or a variant thereof having a single substitution, deletion or insertion of SEQ ID NO: 92; a VH CDR-H3 of SEQ ID NO: 124 or a variant thereof having a single substitution, deletion or insertion of SEQ ID NO: 124; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 61 or a variant thereof having a single substitution, deletion or insertion of SEQ ID NO: 61; a VH CDR-H2 of SEQ ID NO: 93 or a variant thereof having a single substitution, deletion or insertion of SEQ ID NO: 93; a VH CDR-H3 of SEQ ID NO: 125 or a variant thereof having a single substitution, deletion or insertion of SEQ ID NO: 125; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, can, for example, can, for example, can, for example, can, for example, can, for example, can, for example, can, for example, can, for example, can, for example, can, for example, can, for example, can, for example, comprise: a VH CDR-H1 of SEQ ID NO: 62 or a variant thereof having a single substitution, deletion or insertion of SEQ ID NO: 62; a VH CDR-H2 of SEQ ID NO: 94 or a variant thereof having a single substitution, deletion or insertion of SEQ ID NO: 94; a VH CDR-H3 of SEQ ID NO: 126 or a variant thereof having a single substitution, deletion or insertion of SEQ ID NO: 126; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 63 or a variant thereof having a single substitution, deletion or insertion of SEQ ID NO: 63; a VH CDR-H2 of SEQ ID NO: 95 or a variant thereof having a single substitution, deletion or insertion of SEQ ID NO: 95; a VH CDR-H3 of SEQ ID NO: 127 or a variant thereof having a single substitution, deletion or insertion of SEQ ID NO: 127; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 64 or a variant thereof having a single substitution, deletion or insertion of SEQ ID NO: 64; a VH CDR-H2 of SEQ ID NO: 96 or a variant thereof having a single substitution, deletion or insertion of SEQ ID NO: 96; a VH CDR-H3 of SEQ ID NO: 128 or a variant thereof having a single substitution, deletion or insertion of SEQ ID NO: 128; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 65 or a variant thereof having a single substitution, deletion or insertion of SEQ ID NO: 65; a VH CDR-H2 of SEQ ID NO: 97 or a variant thereof having a single substitution, deletion or insertion of SEQ ID NO: 97; a VH CDR-H3 of SEQ ID NO: 129 or a variant thereof having a single substitution, deletion or insertion of SEQ ID NO: 129; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 66 or a variant thereof having a single substitution, deletion or insertion of SEQ ID NO: 66; a VH CDR-H2 of SEQ ID NO: 98 or a variant thereof having a single substitution, deletion or insertion of SEQ ID NO: 98; a VH CDR-H3 of SEQ ID NO: 130 or a variant thereof having a single substitution, deletion or insertion of SEQ ID NO: 130; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 67 or a variant thereof having a single substitution, deletion or insertion of SEQ ID NO: 67; a VH CDR-H2 of SEQ ID NO: 99 or a variant thereof having a single substitution, deletion or insertion of SEQ ID NO: 99; a VH CDR-H3 of SEQ ID NO: 131 or a variant thereof having a single substitution, deletion or insertion of SEQ ID NO: 131; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 68 or a variant thereof having a single substitution, deletion or insertion of SEQ ID NO: 68; a VH CDR-H2 of SEQ ID NO: 100 or a variant thereof having a single substitution, deletion or insertion of SEQ ID NO: 100; a VH CDR-H3 of SEQ ID NO: 132 or a variant thereof having a single substitution, deletion or insertion of SEQ ID NO: 132; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 69 or a variant thereof having a single substitution, deletion or insertion of SEQ ID NO: 69; a VH CDR-H2 of SEQ ID NO: 101 or a variant thereof having a single substitution, deletion or insertion of SEQ ID NO: 101; a VH CDR-H3 of SEQ ID NO: 133 or a variant thereof having a single substitution, deletion or insertion of SEQ ID NO: 133; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 70 or a variant thereof having a single substitution, deletion or insertion of SEQ ID NO: 70; a VH CDR-H2 of SEQ ID NO: 102 or a variant thereof having a single substitution, deletion or insertion of SEQ ID NO: 102; a VH CDR-H3 of SEQ ID NO: 134 or a variant thereof having a single substitution, deletion or insertion of SEQ ID NO: 134; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 71 or a variant thereof having a single substitution, deletion or insertion of SEQ ID NO: 71; a VH CDR-H2 of SEQ ID NO: 103 or a variant thereof having a single substitution, deletion or insertion of SEQ ID NO: 103; a VH CDR-H3 of SEQ ID NO: 135 or a variant thereof having a single substitution, deletion or insertion of SEQ ID NO: 135; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 72 or a variant thereof having a single substitution, deletion or insertion of SEQ ID NO: 72; a VH CDR-H2 of SEQ ID NO: 104 or a variant thereof having a single substitution, deletion or insertion of SEQ ID NO: 104; a VH CDR-H3 of SEQ ID NO: 136 or a variant thereof having a single substitution, deletion or insertion of SEQ ID NO: 136; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 73 or a variant thereof having a single substitution, deletion or insertion of SEQ ID NO: 73; a VH CDR-H2 of SEQ ID NO: 105 or a variant thereof having a single substitution, deletion or insertion of SEQ ID NO: 105; a VH CDR-H3 of SEQ ID NO: 137 or a variant thereof having a single substitution, deletion or insertion of SEQ ID NO: 137; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 74 or a variant thereof having a single substitution, deletion or insertion of SEQ ID NO: 74; a VH CDR-H2 of SEQ ID NO: 106 or a variant thereof having a single substitution, deletion or insertion of SEQ ID NO: 106; a VH CDR-H3 of SEQ ID NO: 138 or a variant thereof having a single substitution, deletion or insertion of SEQ ID NO: 138; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

The antibody or fragment thereof can, for example, comprise: a VH CDR-H1 of SEQ ID NO: 75 or a variant thereof having a single substitution, deletion or insertion of SEQ ID NO: 75; a VH CDR-H2 of SEQ ID NO: 107 or a variant thereof having a single substitution, deletion or insertion of SEQ ID NO: 107; a VH CDR-H3 of SEQ ID NO: 139 or a variant thereof having a single substitution, deletion or insertion of SEQ ID NO: 139; a VL CDR-L1 having the amino acid sequence of any one of SEQ ID NOs: 141-150 or a variant thereof having a single substitution, deletion or insertion of SEQ ID NOs: 141-150; a VL CDR-L2 having the amino acid sequence of any one of SEQ ID NOs: 152-161 or a variant thereof having a single substitution, deletion or insertion of SEQ ID NOs: 152-161; and a VL CDR-L3 having the amino acid sequence of any one of SEQ ID NOs: 163-172 or a variant thereof having a single substitution, deletion or insertion of SEQ ID NOs: 163-172.

In some embodiments, the antibody fragment disclosed herein comprises a heavy chain amino acid sequence selected from SEQ ID NOs: 1-16, 18-33, and 174-184 or a variant thereof having a single substitution, deletion or insertion from any one of SEQ ID NOs: 1-16, 18-33, and 174-184. In some embodiments, the antibody fragment disclosed herein comprises a light chain amino acid sequence selected from SEQ ID NOs: 17, 34-43, and 173 or a variant thereof having a single substitution, deletion or insertion from any one of SEQ ID NOs: 17, 34-43, and 173. In some embodiments, the antibody fragment disclosed herein comprises a heavy chain amino acid sequence selected from SEQ ID NOs: 1-16, 18-33, and 174-184 or a variant thereof having a single substitution, deletion or insertion from any one of SEQ ID NOs: 1-16, 18-33, and 174-184, and a light chain amino acid sequence selected from SEQ ID NOs: 17, 34-43, and 173 or a variant thereof having a single substitution, deletion or insertion from any one of SEQ ID NOs: 17, 34-43, and 173.

Disclosed herein includes a single chain Fab fragment (scFab) of the antibody or the fragment thereof as described herein, wherein the scFab or the scFab fragment thereof comprises a heavy chain of the antibody fragment of a heavy chain variable region (VH) comprising (a) a CDR-H1 comprising an amino acid sequence selected from SEQ ID NOs: 44-75 or a variant thereof having a single substitution, deletion or insertion from any one of SEQ ID NOs: 44-75; (b) a CDR-H2 comprising an amino acid sequence selected from SEQ ID NOs: 76-107 or a variant thereof having a single substitution, deletion or insertion from any one of SEQ ID NOs: 76-107; and a light chain of the antibody fragment of a light chain variable region (VL) comprising (d) a CDR-L1 comprising an amino acid sequence selected from SEQ ID NOs: 140-150 or a variant thereof having a single substitution, deletion or insertion from any one of SEQ ID NOs: 140-150; (e) a CDR-L2 comprising an amino acid sequence selected from SEQ ID NOs: 151-161 or a variant thereof having a single substitution, deletion or insertion from any one of SEQ ID NOs: 151-161; and (f) a CDR-L3 comprising an amino acid sequence selected from SEQ ID NOs: 162-172 or a variant thereof having a single substitution, deletion or insertion from any one of SEQ ID NOs: 162-172, optionally wherein a scFab linker connects a C-terminal of the heavy chain to an N-terminal the light chain.

In some embodiments, the heavy chain and the light chain are connected with a scFab linker, of about 15 to about 45 amino acids, from about 20 to about 40 amino acids, or from 32 to 36 amino acids, or 34 amino acids. In some embodiments, the scFab linker comprises at least 50% of glycine, at least 60% of glycine, at least 70% of glycine, at least 80% of glycine, or at least 90% glycine.

Disclosed herein includes a single chain variable region antibody (scFv) or a scFv fragment thereof, comprises a heavy chain variable region (VH) comprising (a) a CDR-H1 comprising an amino acid sequence selected from SEQ ID NOs: 44-75 or a variant thereof having a single substitution, deletion or insertion from any one of SEQ ID NOs: 44-75; (b) a CDR-H2 comprising an amino acid sequence selected from SEQ ID NOs: 76-107 or a variant thereof having a single substitution, deletion or insertion from any one of SEQ ID NOs: 76-107; and a light chain variable region (VL) comprising (d) a CDR-L1 comprising an amino acid sequence selected from SEQ ID NOs: 140-150 or a variant thereof having a single substitution, deletion or insertion from any one of SEQ ID NOs: 140-150; (e) a CDR-L2 comprising an amino acid sequence selected from SEQ ID NOs: 151-161 or a variant thereof having a single substitution, deletion or insertion from any one of SEQ ID NOs: 151-161; and (f) a CDR-L3 comprising an amino acid sequence selected from SEQ ID NOs: 162-172 or a variant thereof having a single substitution, deletion or insertion from any one of SEQ ID NOs: 162-172, optionally wherein a scFab linker connects a C-terminal of the heavy chain to an N-terminal the light chain.

In some embodiments, the single chain variable region antibody (scFv) or the scFv fragment thereof comprises a scFv linker of about 5 to about 25 amino acids. In some embodiments, the scFv linker comprises at least 50% of glycine, at least 60% of glycine, at least 70% of glycine, at least 80% of glycine, or at least 90% glycine. In some embodiments, the scFv linker comprises an amino acid sequence of SEQ ID NO: 185 or a variant thereof having a single substitution, deletion, or insertion from SEQ ID NO: 185. In some embodiments, an N-terminal of the scFv linker is connected to a C-terminal of the heavy chain variable region and an C-terminal of the linker is connected to an N-terminal of the light chain variable region.

Disclosed herein include the scFv or a scFv fragment thereof comprising an amino acid sequence selected from SEQ ID NOs: 186-196, or a variant thereof having a single substitution, deletion, or insertion from SEQ ID NOs: 186-196.

Disclosed herein include compositions comprising any of the antibodies or fragments thereof provided herein and a pharmaceutically acceptable carrier. Disclosed herein include polynucleotides encoding one or more of the antibody or fragment thereof provided herein. Disclosed herein include isolated cells comprising any of the polynucleotides provided herein. Disclosed herein include compositions comprising any of the polynucleotides and/or an isolated cells provided herein.

There are provided methods of treating or preventing a coronavirus infection in a patient in need thereof, e.g., administering to the patient an effective amount of any of the antibodies or fragments thereof, polynucleotides, isolated cells, and compositions provided herein, or a combination thereof.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

As used herein, a polypeptide “variant” can refer to a biologically active polypeptide having at least about 80% amino acid sequence identity with the corresponding native sequence polypeptide, or fragment thereof. Such variants include, for instance, polypeptides wherein one or more amino acid residues are added, deleted, or substituted. Ordinarily, a variant will have at least about 80% amino acid sequence identity, or at least about 90% amino acid sequence identity, or at least about 95% or more amino acid sequence identity with the native sequence polypeptide, or fragment thereof. The term “variant” as used herein shall have its ordinary meaning, and can also refer to a protein variant as described herein and/or which includes one or more amino acid mutations in the native protein sequence. Optionally, the one or more amino acid mutations include amino acid substitution(s).

The term “isolated” as used herein with respect to cells, proteins, nucleic acids (such as DNA or RNA), refers to molecules separated from other cells, proteins, nucleic acids, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. In some embodiments, an “isolated nucleic acid” refers to nucleic acid fragments which are not naturally occurring as fragments and would not be found in the natural state. The term “isolated” is also used herein to refer to cells or polypeptides which are isolated from other cellular proteins or tissues. Isolated polypeptides can encompass both purified and recombinant polypeptides.

As used herein, the term “recombinant” in the context of polypeptides or polynucleotides refers to a form of the polypeptide or polynucleotide that does not exist naturally, a non-limiting example of which can be created by combining polynucleotides or polypeptides or by combining different polypeptides that would not normally occur together.

As used herein, an “antibody” or “antigen-binding polypeptide” can refer to a polypeptide or a polypeptide complex that specifically recognizes and binds to an antigen (e.g., a spike protein receptor binding domain). An antibody can be a whole antibody and any antigen binding fragment or a single chain thereof. Thus, the term “antibody” includes any protein or peptide-containing molecule that comprises at least a portion of an immunoglobulin molecule having biological activity of binding to the antigen. Examples of such include, but are not limited to, a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, L-RNA aptamers (also known as spiegelmers), and diabodies. The term “antibody fragment” also includes any synthetic or genetically engineered protein that acts like an antibody by binding to a specific antigen to form a complex.

As used herein, a “single-chain variable fragment” or “scFv” refers to a fusion protein of the variable regions of the heavy (V_{H}) and light chains (V_{L}) of immunoglobulins. In some embodiments, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

As used herein, a single chain Fab fragment or scFab, refers to a fusion protein of a heavy chain of the antibody or immunoglobulin and a light chain of the antibody or immunoglobulin. In some embodiments, the heavy chain and the light chain are connected with a short linker peptide, scFab linker, of about 15 to about 45 amino acids, from about 20 to about 40 amino acids, or from 32 to 36 amino acids, or 34 amino acids. The linker can be rich in glycine for flexibility, as well as serine or threonine for solubility wherein a scFab linker connects a C-terminal of the heavy chain to an N-terminal the light chain as described. ScFab molecules are known in the art and are described, e.g., in Michael Hust et al. BMC Biotechnology 2007, 7:14, 15 pages.

As used herein, the term “antibody” encompasses various broad classes of polypeptides that can be distinguished biochemically. Those of skill in the art will appreciate that heavy chains are classified as gamma, mu, alpha, delta, or epsilon (γ, μ, α, δ, or ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight approximately 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, fragments, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies. Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VL chains (i.e. VH CDR1, VH CDR2, VH CDR3, VL CDR1, VL CDR2 and VL CDR3). As used herein, the terms VH CDR1, VH CDR2, VH CDR3, VL CDR1, VL CDR2 and VL CDR3 are used in the present disclosure interchangeably with the terms CDR-H1, CDR-H2, CDR-H3, CDR-L1, CDR-L2, and CDR-L3, respectively. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those of skill in the art can routinely determine which residues comprise a particular CDR given the variable region amino acid sequence of the antibody.

| | Kabat | Chothia |
|---|---|---|
| VH CDR1 (CDR-H1) | 31-35 | 26-32 |
| VH CDR2 (CDR-H2) | 50-65 | 52-58 |
| VH CDR3 (CDR-H3) | 95-102 | 95-102 |
| VL CDR1 (CDR-L1) | 24-34 | 26-32 |
| VL CDR2 (CDR-L2) | 50-56 | 50-52 |
| VL CDR3 (CDR-L3) | 89-97 | 91-96 |

Kabat et al. also defined a numbering system for variable domain sequences that is applicable to any antibody. One of skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: VH CDR1 begins at approximately amino acid 31 (i.e., approximately 9 residues after the first cysteine residue), includes approximately 5-7 amino acids, and ends at the next tryptophan residue. VH CDR2 begins at the fifteenth residue after the end of VH CDR1, includes approximately 16-19 amino acids, and ends at the next arginine or lysine residue. VH CDR3 begins at approximately the thirty third amino acid residue after the end of VH CDR2; includes 3-25 amino acids; and ends at the sequence W-G-X-G, where X is any amino acid. VL CDR1 begins at approximately residue 24 (i.e., following a cysteine residue); includes approximately 10-17 residues; and ends at the next tryptophan residue. VL CDR2 begins at approximately the sixteenth residue after the end of VL CDR1 and includes approximately 7 residues. VL CDR3 begins at approximately the thirty third residue after the end of VL CDR2 (i.e., following a cysteine residue); includes approximately 7-11 residues and ends at the sequence F or W-G-X-G, where X is any amino acid.

Antibodies disclosed herein can be from any animal origin including vertebrates (e.g., birds, reptiles, amphibians, and mammals). In some embodiments, the antibodies are human, murine, donkey, rabbit, goat, guinea pig, camel, llama, horse, or chicken antibodies. In some embodiments, the variable region is condricthoid in origin (e.g., from sharks). In some embodiments, the antibody or fragment thereof is from a mammal.

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG1 molecule and a hinge region derived from an IgG 3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CH2 regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In some embodiments, the target binding region or site is from a non-human source (e.g., mouse or primate) and the constant region is human.

As used herein, the term “binding” refers to a non-covalent interaction between macromolecules (e.g., between a protein and a nucleic acid or between a first protein and a second protein). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (I(d), for example a Kd of, or a Kd less than, 10⁻⁶ M, 10⁻⁷M, 10⁻⁸ M, 10⁻⁹ M, 10⁻¹⁰ M, 10⁻¹¹M, 10⁻¹² M, 10⁻¹³M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd. Binding interactions can also be characterized by an EC50. As used herein, “EC50” can refer to the concentration of an agent (e.g., an antibody or fragment thereof) which produces 50% of the maximal response possible for that agent. As described herein, binding interactions can be characterized by an EC50 of, or an EC50 less than 10 μg/mL, less than 1 μg/mL, less than 0.1 μg/mL, less than 0.01 μg/mL, less than 0.001 μg/mL, or less than 0.0001 μg/mL

The term “IC50,” as used herein, can refer to the half-maximal concentration of an antibody or an antigen- binding fragment thereof, which induces an inhibitory response (e.g., reduced infectivity, e.g., neutralization), either in an in vitro or an in vivo assay, which is 50% of the maximal response, i.e., halfway between the maximal response and the baseline. As used herein, “infectivity” shall have its ordinary meaning, and can also refer to the ability of a virus to enter or exit a cell. As described herein, the antibodies or fragments thereof provided herein can reduce, inhibit, block infectivity of a virus at an IC50 of, e.g., less than 10 μg/mL, less than 1 μg/mL, less than 0.1 μg/mL, or less than 0.01 μg/mL, less than 0.001 μg/mL, or less than 0.0001 μg/mL.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity (e.g., greater binding affinity) for a given epitope than antibody “B,” or antibody “A” may be said to bind to epitope “C” with a higher specificity (e.g., greater binding affinity) than it has for related epitope “D.”

Provided herein are antibodies and fragments thereof with specificity for coronavirus antigens (e.g., spike protein receptor binding domain). Also provided are compositions comprising said antibodies or fragments thereof, and methods for identifying and isolating said antibodies. There are provided methods of treating a subject suffering from a coronavirus infection using any of the antibodies or fragments thereof and compositions described herein. Provided herein are methods to produce therapeutic neutralizing antibody and its fragments that would remain therapeutically effective against Omicron or any future SARS-CoV-2 VOC substitutions.

### Antibodies

The present disclosure provides sarbecovirus specific antibodies or fragments thereof with high affinity and specificity to the virus. In some embodiments, the antibodies herein described are broadly neutralizing and potent. In some embodiments, the antibodies herein described can be used for treating a patient in need thereof, who is suffering from, e.g., a SARS-CoV-2 infection. There are provided, in some embodiments, compositions comprising one or more of the antibody or fragment thereof as described herein.

In some embodiments, the antibodies or fragments thereof disclosed herein contain CDR regions defined in SEQ ID NOs: 49-87 or variants thereof having one, two or three mismatches (e.g., a single substitution, deletion or insertion) in any one of SEQ ID NOs: 49-87.

Disclosed herein include antibodies or fragments thereof. In some embodiments, the antibody or fragment thereof has specificity to one or more sarbecovirus (e.g., one or more SARS-CoV-2) and comprises: (a) a heavy chain variable region CDR-H1 comprising an amino acid sequence selected from SEQ ID NOs: 44-75 or a variant thereof having a single substitution, deletion or insertion from any one of SEQ ID NOs: 44-75; (b) a VH CDR-H2 comprising an amino acid sequence selected from SEQ ID NOs: 76-107 or a variant thereof having a single substitution, deletion or insertion from any one of SEQ ID NOs: 76-107 ; (c) a VH CDR-H3 comprising an amino acid sequence selected from SEQ ID NOs: 108-139 or a variant thereof having a single substitution, deletion or insertion from any one of SEQ ID NOs: 108-139; (d) a light chain LC comprising an amino acid sequence selected from SEQ ID NOs: 17, 34-43 and 173 or a variant thereof having a single substitution, deletion or insertion from any one of SEQ ID NOs: 17, 34-43 and 173; (e) a heavy chain HC comprising an amino acid sequence selected from an amino acid sequence selected from SEQ ID NOs: 1-16, 18-33 and 174-184 or a variant thereof having a single substitution, deletion or insertion from any one of SEQ ID NOs: 1-16, 18-33 and 174-184.

The antibody or fragment thereof can comprise a heavy chain comprising (i) an amino acid sequence selected from SEQ ID NOs: 1-16, 18-33 and 174-184, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 1-16, 18-33 and 174-184, or (iii) an amino acid sequence having one, two or three mismatches relative to an amino acid sequence selected from SEQ ID NOs: 1-16, 18-33 and 174-184.

The antibody or fragment thereof can comprise a light chain comprising (i) an amino acid sequence selected from SEQ ID NOs: 17, 34-43 and 173, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 17, 34-43 and 173, (iii) an amino acid sequence having one, two or three mismatches relative to an amino acid sequence selected from SEQ ID NOs: 17, 34-43 and 173.

The antibody or fragment thereof can comprise a heavy chain variable region CDR-H1 comprising an amino acid sequence selected from SEQ ID NOs: 44-75, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 44-75, or (iii) an amino acid sequence having one, two or three mismatches relative to an amino acid sequence selected from SEQ ID NOs: 44-75.

The antibody or fragment thereof can comprise a heavy chain variable region CDR-H2 comprising an amino acid sequence selected from SEQ ID NOs: 76-107, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 76-107, or (iii) an amino acid sequence having one, two or three mismatches relative to an amino acid sequence selected from SEQ ID NOs: 76-107.

The antibody or fragment thereof can comprise a heavy chain variable region CDR-H3 comprising an amino acid sequence selected from SEQ ID NOs: 108-139, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 108-139, or (iii) an amino acid sequence having one, two or three mismatches relative to an amino acid sequence selected from SEQ ID NOs: 108-139.

The antibody or fragment thereof can comprise a light chain variable region CDR-L1 comprising (i) an amino acid sequence selected from SEQ ID NOs: 140-150, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 140-150, (iii) an amino acid sequence having one, two or three mismatches relative to an amino acid sequence selected from SEQ ID NOs: 140-150.

The antibody or fragment thereof can comprise a light chain variable region CDR-L2 comprising (i) an amino acid sequence selected from SEQ ID NOs: 151-161, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 151-161, (iii) an amino acid sequence having one, two or three mismatches relative to an amino acid sequence selected from SEQ ID NOs: 151-161.

The antibody or fragment thereof can comprise a light chain variable region CDR-L3 comprising (i) an amino acid sequence selected from SEQ ID NOs: 162-172, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 162-172, (iii) an amino acid sequence having one, two or three mismatches relative to an amino acid sequence selected from SEQ ID NOs: 162-172.

The single chain variable region scFv or fragment thereof comprising (i) an amino acid sequence selected from SEQ ID NOs: 186-196, (ii) an amino acid sequence having, having about, having at least, or having at least about 90%, 91%, 92%, 93%, 94%, 95%, 96%, 97%, 98%, or 99% sequence identity to an amino acid sequence selected from SEQ ID NOs: 186-196, (iii) an amino acid sequence having one, two or three mismatches relative to an amino acid sequence selected from SEQ ID NOs: 186-196.

In some embodiments, the antibodies or fragments thereof do not elicit an undesirable (e.g., deleterious) immune response in a subject to be treated, e.g., in a human. In some embodiments, antibodies, fragments, variants, or derivatives thereof of the disclosure are modified to reduce their immunogenicity using techniques recognized in the art. For example, antibodies can be humanized, primatized, deimmunized, or chimeric antibodies.

Binding of the antibodies and fragments thereof as disclosed herein can be assessed by any assay known in the art, including, but not limited to, precipitation assay, agglutination assay, ELISA, surface plasma resonance, western blot, and FACS. In some embodiments, binding can be assessed by an optofluidic system (e.g., Berkeley Light Beacon® Optofluidic System). As described herein, the optofluidic technology can comprise distributing cells within a sample into individual compartments using microfluidic devices, and detecting a signal associated with the subset of cells with the property of interest. The term “enzyme linked immunosorbent assay” (ELISA) as used herein can refer to an antibody-based assay in which detection of the antigen of interest is accomplished via an enzymatic reaction producing a detectable signal. An ELISA can be run as a competitive or non-competitive format.

In some embodiments, the antibody or fragment thereof binds to one or more sarbecoviruses (e.g., one or more of SARS-CoV2 and variants thereof) with an EC50 of less than 10 μg/mL, less than 1 μg/mL, less than 0.1 μg/mL, or less than 0.01 μg/mL as assessed by an optofluidic system and/or an ELISA assay. In some embodiments, the antibody or fragment thereof binds to one or more sarbecovirus (e.g., one or more of SARS-CoV2 and variants thereof) with an EC50 of less than 10 μg/mL, less than 1 μg/mL, less than 0.1 μg/mL, or less than 0.01 μg/mL as assessed by an optofluidic system and/or an ELISA assay.

In some embodiments, the antibody or fragment thereof binds to one or more sarbecoviruses (e.g., one or more of SARS-CoV2 and variants thereof) with an EC50 of about 0.0001 μg/mL to about 10 μg/mL (e.g., 0.0001, 0.0005, 0.001, 0.005, 0.01, 0.05, 0.1, 0.5, 1, 1.5, 2, 3, 4, 5, 6, 7, 8, 9, 10 μg/mL or a number or a range between any two of these values), about 0.0001 μg/mL to about 1 μg/mL (e.g., 0.0001, 0.0005, 0.001, 0.005, 0.01, 0.05, 0.1, 0.5, 1 μg/mL or a number or a range between any two of these values), about 0.0001 μg/mL to about 0.1 μg/mL (e.g., 0.0001, 0.0005, 0.001, 0.005, 0.01, 0.05, 0.1 μg/mL or a number or a range between any two of these values), or about 0.0001 μg/mL to about 0.01 μg/mL (e.g., 0.0001, 0.0005, 0.001, 0.0025, 0.005, 0.0075, 0.01 μg/mL or a number or a range between any two of these values) as assessed by an optofluidic system and/or an ELISA assay. In some embodiments, the antibody or fragment thereof binds to one or more sarbecovirus (e.g., one or more of SARS-CoV2 and variants thereof) with an EC50 of about 0.0001 μg/mL to about 10 μg/mL (e.g., 0.0001, 0.0005, 0.001, 0.005, 0.01, 0.05, 0.1, 0.5, 1, 1.5, 2, 3, 4, 5, 6, 7, 8, 9, 10 μg/mL or a number or a range between any two of these values), about 0.0001 μg/mL to about 1 μg/mL (e.g., 0.0001, 0.0005, 0.001, 0.005, 0.01, 0.05, 0.1, 0.5, 1 μg/mL or a number or a range between any two of these values), about 0.0001 μg/mL to about 0.1 μg/mL (e.g., 0.0001, 0.0005, 0.001, 0.005, 0.01, 0.05, 0.1 μg/mL or a number or a range between any two of these values), or about 0.0001 μg/mL to about 0.01 μg/mL (e.g., 0.0001, 0.0005, 0.001, 0.0025, 0.005, 0.0075, 0.01 μg/mL or a number or a range between any two of these values) as assessed by an optofluidic system and/or an ELISA assay.

As disclosed herein, sarbecoviruses can be SARS-CoV-2 and variants thereof, RsSTT200 and variants thereof, Pang17 and variants thereof, RaTG13 and variants thereof, SARS-CoV and variants thereof, WIV1 and variants thereof, SHC014 and variants thereof, LyRa3 and variants thereof, C028 and variants thereof, Rs4081 and variants thereof, RmYN02 and variants thereof, Rf1 and variants thereof, Yun11 and variants thereof, BM4831 BD and variants thereof, BtKY72 and variants thereof, and Khosta2 and variants thereof.

In some embodiments, the antibodies and fragments thereof comprise potent and/or broad neutralization activities against, e.g., one or more coronaviruses. “Potency” as used herein can refer to a measure of how effective an antibody or fragment thereof is at producing the desired response (e.g., inhibiting infectivity) and can be expressed in terms of the concentration (e.g., IC50) which produces a particular level of the response attainable. Broadly neutralizing antibodies are antibodies that can neutralize coronaviruses from two or more taxonomic groups (e.g., subfamily, genus, subgenus, species, and/or strain) of coronavirus. Broadly neutralizing response can also be referred to as heterologous neutralizing response. In some embodiments, the methods described herein can elicit broadly neutralizing antibodies that neutralize one or more coronaviruses from a subfamily, genus, subgenus, species, and/or strain that differ from the subfamily, genus, subgenus, species, and/or strain of the coronaviruses from which the coronavirus antigens are derived to produce the multivalent nanoparticles (See, “Methods for identifying antibodies or fragments thereof that specifically bind coronavirus antigens” below).

The term “neutralizing,” as used herein, in relation to the antibodies of the disclosure refers to antibodies that are capable of preventing, reducing or inhibiting infection of a cell by the virus, by neutralizing, inhibiting, or reducing its biological effect and/or reducing the infectious titer of the virus, regardless of the mechanism by which neutralization is achieved. Neutralization can, e.g., be achieved by inhibiting the attachment or adhesion of the virus to the cell surface, or by inhibition of the fusion of viral and cellular membranes following attachment of the virus to the target cell, and the like (e.g., by interfering with ACE2 binding). Neutralization potencies can be determined by any method known in the art. In some embodiments, reduced infectivity and IC50 values can be determined by, e.g., a pseudovirus neutralization assay as described in the Example section.

In some embodiments, the antibody or fragment thereof inhibits infectivity of one or more sarbecoviruses (e.g., one or more of SARS-CoV2 and variants thereof) with an IC50 less than 10 μg/mL, less than 1 μg/mL, less than 0.1 μg/mL or less than 0.01 μg/mL. In some embodiments, IC50 is measured by a pseudovirus neutralization assay. In some embodiments, the antibody or fragment thereof inhibits infectivity of a virus comprising a sarbecovirus spike protein receptor binding domain (RBD) with an IC50 of about 0.0001 μg/mL to about 10 μg/mL (e.g., 0.0001, 0.0005, 0.001, 0.005, 0.01, 0.05, 0.1, 0.5, 1, 1.5, 2, 3, 4, 5, 6, 7, 8, 9, 10 μg/mL or a number or a range between any two of these values), about 0.0001 μg/mL to about 1 μg/mL (e.g., 0.0001, 0.0005, 0.001, 0.005, 0.01, 0.05, 0.1, 0.5, 1 μg/mL or a number or a range between any two of these values), about 0.0001 μg/mL to about 0.1 μg/mL (e.g., 0.0001, 0.0005, 0.001, 0.005, 0.01, 0.05, 0.1 μg/mL or a number or a range between any two of these values), or about 0.0001 μg/mL to about 0.01 μg/mL (e.g., 0.0001, 0.0005, 0.001, 0.025, 0.005, 0.075, 0.01 μg/mL or a number or a range between any two of these values). In some embodiments, IC50 is measured by a pseudovirus neutralization assay. The pseudovirus neutralization assay can comprise target cells expressing hACE2 receptor protein.

In some embodiments, the antibody or fragment thereof inhibits infectivity of one or more sarbecoviruses (e.g., one or more of SARS-CoV2 and variants thereof) with an IC50 less than 10 μg/mL, less than 1 μg/mL, less than 0.1 μg/mL, or less than 0.01 μg/mL. In some embodiments, the antibody or fragment thereof inhibits infectivity of the virus(es) with an IC50 of about 0.0001 μg/mL to about 10 μg/mL (e.g., 0.0001, 0.0005, 0.001, 0.005, 0.01, 0.05, 0.1, 0.5, 1, 1.5, 2, 3, 4, 5, 6, 7, 8, 9, 10 μg/mL or a number or a range between any two of these values), about 0.0001 μg/mL to about 1 μg/mL (e.g., 0.0001, 0.0005, 0.001, 0.005, 0.01, 0.05, 0.1, 0.5, 1 μg/mL or a number or a range between any two of these values), about 0.0001 μg/mL to about 0.1 μg/mL (e.g., 0.0001, 0.0005, 0.001, 0.005, 0.01, 0.05, 0.1 μg/mL or a number or a range between any two of these values), or about 0.001 μg/mL to about 0.01 μg/mL (e.g., 0.001, 0.005, 0.01 μg/mL or a number or a range between any two of these values).

Disclosed herein include compositions comprising any of the antibodies or fragments thereof provided herein and a pharmaceutically acceptable carrier. Also provided are polynucleotides encoding one or more of the antibody or fragment thereof provided herein. Disclosed herein include isolated cells comprising any of the polynucleotides provided herein. Compositions comprising any of the polynucleotides and/or an isolated cells are provided herein.

The present disclosure provides isolated polynucleotides or nucleic acid molecules encoding the antibodies, fragments, variants or derivatives thereof of the disclosure. The polynucleotides of the present disclosure can encode the heavy and light chain variable regions of the antibodies, fragments, variants or derivatives thereof on the same polynucleotide molecule or on separate polynucleotide molecules. In some embodiments, the polynucleotides of the present disclosure can encode portions of the heavy and light chain variable regions of the antibodies (e.g., the CDR regions), fragments, variants or derivatives thereof on the same polynucleotide molecule or on separate polynucleotide molecules.

Methods of making antibodies are well known in the art and described herein. For example, polynucleotides encoding desired antibodies can be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA can be placed into expression vectors, which are then transfected into antibody-producing cells including prokaryotic or eukaryotic host cells such as E. coli cells, simian COS cells, Chinese Hamster Ovary (CHO) cells or myeloma cells that do not otherwise produce immunoglobulins. The isolated DNA can be used to clone constant and variable region sequences for the manufacture antibodies as described in Newman et al., U.S. Pat. No. 5,658,570 which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As described herein, transformed cells expressing the desired antibody can be grown up in relatively large quantities to provide clinical and commercial supplies of the immunoglobulin.

In some embodiments, mutations can be introduced in the nucleotide sequence encoding an antibody of the present disclosure using standard techniques known to those of skill in the art, including, but not limited to, site-directed mutagenesis and PCR-mediated mutagenesis which result in amino acid substitutions.

In some embodiments, the antibodies, fragments, variants, or derivatives thereof can further comprise a chemical moiety not naturally associated with an antibody. For example, the antibody or fragment thereof can comprise a flexible linker or can be modified to add a functional moiety such as a detectable label. The antibodies, fragments, variants, or derivatives thereof can be modified, i.e., by the covalent or non-covalent attachment of a chemical moiety to the antibody such that the attachment does not interfere or prevent the antibody from binding to the epitope. The chemical moiety can be conjugated to an antibody using any technique known in the art.

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

In some embodiments, the prepared antibodies do not elicit a deleterious immune response in the subject to be treated, e.g., in a human. In one embodiment, antigen-binding polypeptides, variants, or derivatives thereof of the disclosure are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 57:6851-6855 (1984); Morrison et al., Adv. Immunol. 44:65-92 (1988); Verhoeyen et al., Science 239:1534-1536 (1988); Padlan, Molec. Immun. 25:489-498 (1991); Padlan, Molec. Immun. 31:169-217 (1994), and U.S. Pat. Nos.: 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T-cell epitopes (see, e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of the antibodies or fragments thereof of the present disclosure can be determined by in vitro assays such as immunoprecipitation, radioimmunoassay (MA) or enzyme-linked immunoabsorbent assay (ELISA).

Alternatively, techniques described for the production of single-chain units (U.S. Pat. No. 4,694,778; Bird, Science 242:423-442 (1988); Huston et al., Proc. Natl. Acad. Sci. USA 55:5879- 5883 (1988); and Ward et al., Nature 334:544-554 (1989)) can be adapted to produce single-chain units of the present disclosure. Single-chain units are formed by linking the heavy and light chain fragments of the Fv region via an amino acid bridge, resulting in a single-chain fusion peptide. Techniques for the assembly of functional Fv fragments in E. coli may also be used (Skerra et al., Science 242: 1038-1041 (1988)).

Examples of techniques which can be used to produce single-chain Fvs (scFvs) and antibodies include those described in U.S. Pat. Nos. 4,946,778 and 5,258,498; Huston et al., Methods in Enzymology 203:46-88 (1991); Shu et al., Proc. Natl. Sci. USA 90:1995-1999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties.

Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety).

Completely human antibodies can be particularly desirable for therapeutic treatment of human patients. Human antibodies can be made by a variety of methods known in the art including phage display methods using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies can be engaged to provide human antibodies directed against a selected antigen using technology similar to that described above.

Completely human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 72:899-903 (1988). See also, U.S. Pat. No. 5,565,332, which is incorporated by reference in its entirety).

Additionally, using routine recombinant DNA techniques, one or more of the CDRs of the antibodies of the present disclosure, may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present disclosure and within the skill of the art.

In addition, techniques developed for the production of “chimeric antibodies” (Morrison et al., Proc. Natl. Acad. Sci. USA:851-855 (1984); Neuberger et al., Nature 372:604-608 (1984); Takeda et al., Nature 314:452-454 (1985)) by splicing genes from a mouse antibody molecule, of appropriate antigen specificity, together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

Alternatively, antibody-producing cell lines may be selected and cultured using techniques well known to the skilled artisan. Such techniques are described in a variety of laboratory manuals and primary publications. In this respect, techniques suitable for use in the disclosure as described below are described in Current Protocols in Immunology, Coligan et al., Eds., Green Publishing Associates and Wiley-Interscience, John Wiley and Sons, New York (1991) which is herein incorporated by reference in its entirety, including supplements.

Additionally, standard techniques known to those of skill in the art can be used to introduce mutations in the nucleotide sequence encoding an antibody of the present disclosure, including, but not limited to, site-directed mutagenesis and PCR-mediated mutagenesis which result in amino acid substitutions. Preferably, the variants (including derivatives) encode fewer than 50 amino acid substitutions, fewer than 40 amino acid substitutions, fewer than 30 amino acid substitutions, fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the reference variable heavy chain region, CDR-H1, CDR-H2, CDR-H3, variable light chain region, CDR-L1, CDR-L2, and/or CDR-L3. In some embodiments, one or more mutations are introduced randomly along all or part of the coding sequence, such as by saturation mutagenesis, and the resultant mutants can be screened for biological activity to identify mutants that retain activity.

### Methods of Treating Coronavirus Infections

Disclosed herein include methods of treating or preventing a coronavirus infection in a patient in need thereof, e.g., administering to the patient an effective amount of any of the antibodies or fragments thereof, polynucleotides, isolated cells, and compositions provided herein, or a combination thereof. The compositions disclosed herein can be employed in a variety of therapeutic or prophylactic applications to treat or prevent a coronavirus infection in a subject in need, and/or to treat or prevent a disease or disorder caused by a coronavirus in a subject in need.

As used herein, the term “treatment” or “treat” refers to an intervention made in response to a disease, disorder or physiological condition (e.g., a coronavirus infection) manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

Signs and symptoms manifesting a disease or disorder caused by a coronavirus infection can include, but not limited to, fever, cough, tiredness, a loss of taste or smell, shortness of breath or difficulty breathing, muscle aches, chills, sore throat, runny nose, headache, chest pain, pink eye (conjunctivitis), nausea, vomiting, diarrhea, rash, pneumonia and acute respiratory distress syndrome. Diseases or disorders caused by a coronavirus infection may also include severe complications including but not limited to heart disorders including arrhythmias, cardiomyopathy, acute cardiac injury, coagulation disorders including thromboembolism and pulmonary emboli, disseminated intravascular coagulation (DIC), hemorrhage, and arterial clot formation, Guillain-Barré syndrome, sepsis, shock, multiorgan failure, and multisystem inflammatory syndrome, and any combination thereof.

The terms “subject”, “subject in need”, and “individual” as used herein refer to an animal and in particular higher animals and in particular vertebrates such as mammals and more particularly human beings. In some embodiments, the subject or individual has been exposed to a coronavirus. The term “exposed” indicates the subject has come in contact with a person or an animal that is known to be infected with a coronavirus. In some embodiments, a subject in need can be a healthy subject exposed to or at risk of being exposed to a coronavirus. In some embodiments, subjects in need include those already suffering from the disease or disorder caused by a coronavirus infection or those diagnosed with a coronavirus infection.

Accordingly, the composition (e.g., comprising an antibody or fragment thereof described herein) can be administered in advance of any symptom, for example, in advance of a coronavirus infection. The composition can also be administered at or after the onset of a symptom of disease or infection, for example, after development of a symptom of infection or after diagnosis of the infection.

The phrase “therapeutically effective amount” as used herein means the amount of an antibody or fragment thereof disclosed herein which is effective for producing some desired therapeutic effect and/or generating a desired response, such as reduce or eliminate a sign or symptom of a condition or disease, such as pneumonia, at a reasonable benefit/risk ratio. The therapeutically effective amount also varies depending upon neutralization potency, the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of a disclosed composition for the treatment of that disease or disorder is the amount necessary to achieve at least a 20% reduction in that measurable parameter.

In some embodiments, a therapeutically effective amount is used to inhibit coronavirus replication or to measurably alleviate outward symptoms of the viral infection or inhibiting further development of the disease, condition, or disorder. In some embodiments, a therapeutically effective amount is an amount that prevents one or more signs or symptoms that can be caused by a coronavirus infection. In some embodiments, a therapeutically effective amount is an amount that prevents one or more signs or symptoms of a particular disease or condition from developing, such as one or more signs or symptoms associated with coronavirus infections.

A therapeutically effective amount of a composition herein described can be estimated from data obtained from cell culture assays and further determined from data obtained in animal studies, followed up by human clinical trials. For example, toxicity and therapeutic efficacy of the compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

The compositions herein described can be administered using techniques well known to those skilled in the art, such as injection, inhalation or insulation or by oral, parenteral or rectal administration. The composition can be administered by means including, but not limited to, traditional syringes and needleless injection devices. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the composition herein described can be formulated in aqueous solutions, preferably in physiologically compatible buffers such as Hanks' solution, Ringer's solution, or physiological saline buffer.

The antibodies and compositions thereof can be administered to a subject systematically. The wording “systemic administration” as used herein indicates any route of administration by which a composition is brought in contact with the body of the individual, so that the resulting composition location in the body is systemic (i.e., not limited to a specific tissue, organ, or other body part where the antibody or fragment thereof is administered). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

The frequency of administration can vary. A subject can receive dosing for a period of about, less than about, or greater than about 1, 2, 3, 4, 5, 6, 7, 8, 9, 10, 11, 12, 13, 14, or more days, weeks, or months. The compositions can be administered periodically. For example, the compositions can be administered one, two, three, four times a day, or even more frequent. The subject can be administered every 1, 2, 3, 4, 5, 6, or 7 days. In some embodiments, the compositions are administered three times daily. The period of treatment can be for about 1, 2, 3, 4, 5, 6, 7, 8, or 9 days, 2 weeks, 1-11 months, or 1 year, 2 years, 5 years, or even longer. In some embodiments disclosed herein, the dosages that are administered to a subject can change or remain constant over the period of treatment. For example, the daily dosing amounts can increase or decrease over the period of administration. Therefore, the composition can be administered to the subject in need two or more times. A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the compositions include but are not limited to intradermal, intramuscular, intraperitoneal, intravenous, subcutaneous, intranasal, epidural, and oral routes. The antibodies or fragments thereof or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracisternally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion. Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It can be desirable to administer the antibodies or fragments thereof or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The antibody or fragment thereof or composition can be delivered in a controlled release system. For example, a pump may be used (see Sefton, 1987, CRC Crit. Ref. Biomed. Eng. 14:201; Buchwald et al., 1980, Surgery 88:507; Saudek et al., 1989, N. Engl. J. Med. 321:574). As another example, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another example, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Some other non-limiting examples of controlled release systems are described in Langer (1990, Science 249:1527-1533).

In some embodiments where the composition of the disclosure comprises a nucleic acid or polynucleotide encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The amount of the antibodies of the disclosure which will be effective in the treatment, inhibition and prevention of a coronavirus infection can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies or fragments thereof of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

The methods for treating a coronavirus infection comprising administration of an antibody, variant, or derivative thereof of the disclosure are typically tested in vitro, and then in vivo in an acceptable animal model, for the desired therapeutic or prophylactic activity, prior to use in humans. Suitable animal models, including transgenic animals, are well known to those of ordinary skill in the art. For example, in vitro assays to demonstrate the therapeutic utility of antigen-binding polypeptide described herein include the effect of an antigen-binding polypeptide on a cell line or a patient tissue sample. The effect of the antibody or fragment thereof on the cell line and/or tissue sample can be determined utilizing techniques known to those of skill in the art, such as the assays disclosed elsewhere herein. In accordance with the disclosure, in vitro assays which can be used to determine whether administration of a specific antigen-binding polypeptide is indicated, include in vitro cell culture assays in which a patient tissue sample is grown in culture, and exposed to or otherwise administered a compound, and the effect of such compound upon the tissue sample is observed.

Various delivery systems are known and can be used to administer an antibody of the disclosure or a polynucleotide encoding an antibody of the disclosure, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc.

In some embodiments, a method for treating or preventing a coronavirus infection in a subject in need thereof is disclosed, the method comprising administering to the subject a pharmaceutically effective amount of the composition herein described, thereby treating or preventing the coronavirus infection in the subject. In some embodiments, administering the composition comprising an antibody or fragment thereof disclosed herein results in treating or preventing infection caused by a coronavirus different from the coronavirus used to identify and isolate said antibody or fragment thereof. In some embodiments, administering the composition results in treating or preventing infection caused by additional coronaviruses different from the coronaviruses from which the two or more coronavirus antigens are derived to produce the antibody or fragment thereof. In some embodiments, administering the composition results in treating or preventing infection caused by the coronaviruses from which the sarbecovirus are derived to identify and produce the antibody or fragment thereof.

In some embodiments, a method of treating or preventing a disease or disorder caused by a coronavirus infection in a subject in need thereof is disclosed, the method comprising administering to the subject a pharmaceutically effective amount of the compositions herein described, thereby treating or preventing the disease or disorder caused by the coronavirus infection in the subject. In some embodiments, administering the composition results in treating or preventing the disease or disorder caused by a coronavirus different from the coronavirus used to identify the antibody or fragment thereof comprising the composition. In some embodiments, administering the composition results in treating or preventing the disease or disorder caused by additional coronaviruses different from the coronaviruses from which the coronavirus antigens (e.g., sarbecovirus RBDs) are derived to produce the composition. In some embodiments, administering the composition results in treating or preventing the disease or disorder caused by the coronaviruses from which the two or more different coronavirus antigens are derived to produce the composition.

In some embodiments, the composition can be used for treating and preventing a broad spectrum of coronavirus infections or a disease or disorder caused by such infections due to the broadly neutralizing and potent activities of the antibodies disclosed herein. For example, the composition herein described can neutralize one or more coronaviruses from a subfamily, genus, subgenus, species, and/or strain that differ from the subfamily, genus, subgenus, species, and/or strain of the coronaviruses from which the coronavirus antigens are derived to produce the composition.

The antibodies and compositions described herein can treat or prevent infection by an antigenically divergent coronavirus. Therefore, in some embodiments, a composition made using the antibodies or fragments thereof described herein can be used to treat an infection resulting from emerging coronaviruses and variants thereof. For example, an antibody identified and isolated from B-cells isolated from an animal immunized with coronavirus antigens of SARS-CoV2 and SHC014 can protect an individual against infection by antigenically divergent coronavirus strains of Sarbecovirus and by diverging coronavirus strains of the future. The coronavirus can be a coronavirus in the genus of Alpha-coronavirus, Beta-coronavirus, or both. The coronavirus can be a coronavirus of the subgenus Sarbecovirus.

The coronavirus can be SARS-CoV-2 or a variant thereof, B.1.1.7 (20I/501Y.V1), B.1.351 (20H/501Y.V2), P.1 (20J/501Y.V3), RsSTT200 or a variant thereof, Pang17 or a variant thereof, RaTG13 or a variant thereof, SARS-CoV or a variant thereof, WIV1 or a variant thereof, SHC014 or a variant thereof, LyRa3 or a variant thereof, C028 or a variant thereof, Rs4081 or a variant thereof, RmYN02 or a variant thereof, Rf1 or a variant thereof, Yun11 or a variant thereof, BM4831 or a variant thereof, BtKY72 or a variant thereof, or Khosta2 or a variant thereof. The coronavirus can comprise a SARS-CoV-2 variant of concern, variant of interest, or both. The SARS-CoV-2 variant can be SARS-CoV-2 Beta and variants thereof, SARS-CoV-2 Delta and variants thereof, or SARS-CoV-2 Omicron and variants thereof. The compositions described herein can be used to protect a subject against infection by heterologous coronaviruses (e.g., coronaviruses of different taxonomic groups). For example, a composition can comprise antibodies or fragments thereof capable of preventing or treating the subject for infection by not only WIV1, Rf1, RmYN02 and pang17 at a comparable magnitude, but also coronavirus SARS-CoV2, SHC014, SARS-CoV, Yun 11, BM-4831 and BtKY72.

### Co-Therapies

The method can comprise administering to the patient a second therapeutic agent. The second therapeutic agent can comprise an anti-viral compound, an immunosuppressant, an antibody, or any combination thereof. The second therapeutic agent can comprise remdesivir, molnupiravir, tocilizumab, favipiravir, merimepodib, artesunate, favipiravir, ribavirin, EIDD-2801, niclosamide, nitazoxanide, oseltamivir, AT-527, paxlovid, regdanvimab, ramdicivir, baricitinib, imatinib, casirivimab, imdevimab, bemcentinib, bamlanivimab, etesevimab, sotrovimab, leronlimab, bebtelovimab, cilgavimab, IMU-838, oseltamivir, or dexamethasone.

As disclosed herein, co-administration of particular ratios and/or amounts of the antibodies or fragments thereof or a composition comprising said antibodies or fragments thereof, and the second therapeutic agent can result in synergistic effects in reducing, treating, or preventing a coronavirus infection. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be about, or greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is an enhancement in reducing, treating, or preventing a coronavirus infection and that efficacy is increased to a greater degree as compared to the sum of the effects of administering each component, determined as if each component exerted its effect independently, also referred to as the predicted additive effect herein. For example, if a composition comprising component (a) yields an effect of a 20% improvement in e.g., treating a coronavirus infection and a composition comprising component (b) yields an effect of 50% improvement in treating a coronavirus infection, then a composition comprising both component (a) and component (b) would have a synergistic effect if the combination composition's effect on treating a coronavirus infection was greater than 70%.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least about 20, 50, 75, 90, 100, 150, 200 or 300% greater than the predicted additive effect. In some embodiments, the synergistic effect can be at least about 0.2, 0.5, 0.9, 1.1, 1.5, 1.7, 2, or 3 fold greater than the predicted additive effect. In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements in the reduction and prevention of coronavirus infection.

### Compositions

Disclosed herein include compositions comprising any of the antibodies or fragments thereof provided herein and a pharmaceutically acceptable carrier. Disclosed herein include compositions comprising any of the polynucleotide and/or isolated cell provided herein. In some embodiments, the composition further includes a second therapeutic agent (e.g., an anti-viral agent). The second therapeutic agent can comprise an anti-viral compound, an immunosuppressant, an antibody, or any combination thereof. The second therapeutic agent can comprise remdesivir, molnupiravir, tocilizumab, favipiravir, merimepodib, artesunate, favipiravir, ribavirin, EIDD-2801, niclosamide, nitazoxanide, oseltamivir, AT-527, paxlovid, regdanvimab, ramdicivir, baricitinib, imatinib, casirivimab, imdevimab, bemcentinib, bamlanivimab, etesevimab, sotrovimab, leronlimab, bebtelovimab, cilgavimab, IMU-838, oseltamivir, or dexamethasone.

In some embodiments, the term “pharmaceutically acceptable” means approved by a regulatory agency of the Federal or a state government or listed in the U.S. Pharmacopeia or other generally recognized pharmacopeia for use in animals, and more particularly in humans. A “pharmaceutically acceptable carrier” is generally a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

The composition can be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The antibodies of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Kits

The compositions described herein can be provided as components of a kit. The kit can include compositions of the present disclosure as well components for making such compositions. The kit can include, e.g., primers, nucleic acid molecules, expression vectors, nucleic acid constructs encoding protein antigens and/or particle-forming subunits described herein, cells, buffers, substrates, reagents, administration means (e.g., syringes), and instructions for using any of said components. Kits can include compositions comprising one or more antibodies or fragments thereof disclosed herein. It should be appreciated that a kit may comprise more than one container comprising any of the aforementioned, or related, components. For example, certain parts of the kit may require refrigeration, whereas other parts can be stored at room temperature. Thus, a kit can comprise components sold in separate containers by one or more entity, with the intention that the components contained therein be used together.

### EXAMPLES

Some aspects of the embodiments discussed above are disclosed in further detail in the following examples, which are not in any way intended to limit the scope of the present disclosure.

In the examples described below, the pseudo virus neutralization assays were performed to evaluating the antibodies. In the assays, the gene encoding S protein from SARS-CoV-2 variants (without the C-terminal 21 amino acids in the cytoplasmic tail) was co-transfected with Env-deficient HIV backbone to create pseudotyped lentiviral particles. For neutralization assays, serially diluted purified IgG was incubated with SARS-CoV-2 pseudotyped virus for 1 hour at 37° C. After incubation with 293TACE2 target cells for 48 hours at 37° C., cells were lysed and luciferase activity was measured using Britelite Plus (Perkin Elmer). Relative luminescence units (RLUs) were normalized to values derived from cells infected with pseudotyped virus in the absence of antibody. Data are fit to a 5-parameter nonlinear regression in AntibodyDatabase.

### Example 1. Identification of Synthetic Antibody Variants V1 to V7 and V9-V12

Synthetic antibodies, antibody variants and antibody fragments against SARS-CoV-2 were identified. Antibody variants V1 to V7 and V9-V12 each comprise two light chains of L1 of the amino sequences of SEQ ID NO 173 and two heavy chains selected from V1 to V7 and V9 to V12 of the amino sequences of SEQ ID NOS. 174 to 184, respectively.

| SEQ | | |
|---|---|---|
| ID | Antibody Variant | |
| NO | Full Chain | Protein Sequence |
| 173 | L1-Light Chain | MGWSCIILFLVATATGVHSDIQLTQSPSSLSASVGDRVTITCRAS |
| | | QSISSYLNWYQQKPGKAPKLLIYAASSLQSGVPSRFSGSGSGTD |
| | | FTLTISSLQPEDFATYYCQQSYSTPRTFGQGTKVEIKRTVAAPSV |
| | | FIFPPSDEQLKSGTASVVCLLNNFYPREAKVQWKVDNALQSGN |
| | | SQESVTEQDSKDSTYSLSSTLTLSKADYEKHKVYACEVTHQGL |
| | | SSPVTKSFNRGEC |
| 174 | V1-Heavy Chain | MGWSCIILFLVATATGVHSEVQLVESGGGVVQPGRSLRLSCAA |
| | | SGFPFSIFGMHWVRQAPGKGLEWVAVISYDGSHKYYADSVKG |
| | | RFTISRDNSKNTLYLQMNSLRAEDTAVYYCAKEGRPDDIVHVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 175 | V2-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRGDDIVVVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 176 | V3-Heavy Chain | MGWSCIILFLVATATGVHSEVQLVESGGGVVQPGRSLRLSCAA |
| | | SGFPFSIYGMHWVRQAPGKGLEWVAVISYDGSNKYYADSVKG |
| | | RFTISRDNSKNTLYLQMNSLRAEDTAVYYCAKEGRPDDIVRVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 177 | V4-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRPSDIVHVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 178 | V5-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRPSDIVGVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 179 | V6-Heavy Chain | MGWSCIILFLVATATGVHSEVQLVESGGGVVQPGRSLRLSCAA |
| | | SGFPFSIFGMHWVRQAPGKGLEWVAVISYDGSHKYYADSVKG |
| | | RFTISRDNSKNTLYLQMNSLRAEDTAVYYCAKEGRPSDIVHVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 180 | V7-Heavy Chain | MGWSCIILFLVATATGVHSEVQLVESGGGVVQPGRSLRLSCAA |
| | | SGFPFSIFGMHWVRQAPGKGLEWVAVISYDGSHKYYADSVKG |
| | | RFTISRDNSKNTLYLQMNSLRAEDTAVYYCAKEGRPDDIVRVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 181 | V9-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRSSDIVLVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 182 | V10-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRPSDIWHVS |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 183 | V11-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRPDDIVHVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 184 | V12-Heavy Chain | MGWSCIILFLVATATGVHSEVQLVESGGGVVQPGRSLRLSCAA |
| | | SGFPFSIYGMHWVRQAPGKGLEWVAVISYDGSHKYYADSVKG |
| | | RFTISRDNSKNTLYLQMNSLRAEDTAVYYCAKEGRPDDIVRVV |
| | | AFDYWGQGTLVTVSSASTKGPSVFPLAPSSKSTSGGTAALGCL |
| | | VKDYFPEPVTVSWNSGALTSGVHTFPAVLQSSGLYSLSSVVTVP |
| | | SSSLGTQTYICNVNHKPSNTKVDKRVEPKSCDKTHTCPPCPAPE |
| | | LLGGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |

### Example 2. Identification of Single Chain Variable Fragments scFv Variant 1 (V1) to scFv variant 7 (V7) and scFv Variant 9 (V9) to scFv Variant 12 (V12)

The variable regions of each of the full-length antibodies were expressed as single chain variable fragments (scFv) linking the variable region of the Heavy chain to the variable region of the Light chain using the peptide linker SGGGGSGGGGSGGGG (SEQ ID NO: 185). The variable region of the Light chain is the same for each of the single chain variable fragments scFv variant 1 (V1) to scFv variant 7 (V7) and scFv variant 9 (V9) to scFv variant 12 (V12) listed below in the present example. Single chain variable fragments scFv variant 1 (V1) to scFv variant 7 (V7) and scFv variant 9 (V9) to scFv variant 12 (V12) each comprise the same variable region of the light chain of L1 of the amino acid sequence SEQ ID NO: 173 and a variable region of the heavy chains selected from V1 to V7 and V9 to V12 of the amino acid sequences of SEQ ID NOS: 174 to 184, respectively. The resulting corresponding single chain variable fragments scFv variant 1 (V1) to scFv variant 7 (V7), scFv variant 9 (V9) to scFv variant 12 (V12) of the amino acid sequences of SEQ ID NOS: 186 to 196 are listed in the table below.

| SEQ ID | single chain variable | |
|---|---|---|
| NO | fragments (scFv) | Protein Sequence |
| 186 | scFv variant 1 (V1) | MGWSCIILFLVATATGVHSEVQLVESGGGVVQPGRSLRLSC |
| | | AASGFPFSIFGMHWVRQAPGKGLEWVAVISYDGSHKYYADS |
| | | VKGRFTISRDNSKNTLYLQMNSLRAEDTAVYYCAKEGRPDD |
| | | IVHVVAFDYWGQGTLVTVSSGGGGSGGGGSGGGGSDIQLTQ |
| | | SPSSLSASVGDRVTITCRASQSISSYLNWYQQKPGKAPKLLIY |
| | | AASSLQSGVPSRFSGSGSGTDFTLTISSLQPEDFATYYCQQSY |
| | | STPRTFGQGTKVEIK |
| 187 | scFv variant 2 (V2) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRGD |
| | | DIVVVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 188 | scFv variant 3 (V3) | MGWSCIILFLVATATGVHSEVQLVESGGGVVQPGRSLRLSC |
| | | AASGFPFSIYGMHWVRQAPGKGLEWVAVISYDGSNKYYAD |
| | | SVKGRFTISRDNSKNTLYLQMNSLRAEDTAVYYCAKEGRPD |
| | | DIVRVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 189 | scFv variant 4 (V4) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRPS |
| | | DIVHVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 190 | scFv variant 5 (V5) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRPS |
| | | DIVGVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 191 | scFv variant 6 (V6) | MGWSCIILFLVATATGVHSEVQLVESGGGVVQPGRSLRLSC |
| | | AASGFPFSIFGMHWVRQAPGKGLEWVAVISYDGSHKYYADS |
| | | VKGRFTISRDNSKNTLYLQMNSLRAEDTAVYYCAKEGRPSD |
| | | IVHVVAFDYWGQGTLVTVSSGGGGSGGGGSGGGGSDIQLTQ |
| | | SPSSLSASVGDRVTITCRASQSISSYLNWYQQKPGKAPKLLIY |
| | | AASSLQSGVPSRFSGSGSGTDFTLTISSLQPEDFATYYCQQSY |
| | | STPRTFGQGTKVEIK |
| 192 | scFv variant 7 (V7) | MGWSCIILFLVATATGVHSEVQLVESGGGVVQPGRSLRLSC |
| | | AASGFPFSIFGMHWVRQAPGKGLEWVAVISYDGSHKYYADS |
| | | VKGRFTISRDNSKNTLYLQMNSLRAEDTAVYYCAKEGRPDD |
| | | IVRVVAFDYWGQGTLVTVSSGGGGSGGGGSGGGGSDIQLTQ |
| | | SPSSLSASVGDRVTITCRASQSISSYLNWYQQKPGKAPKLLIY |
| | | AASSLQSGVPSRFSGSGSGTDFTLTISSLQPEDFATYYCQQSY |
| | | STPRTFGQGTKVEIK |
| 193 | scFv variant 9 (V9) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRSS |
| | | DIVLVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 194 | scFv variant 10 (V10) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRPS |
| | | DIWHVSAFDYWGQGTLVTVSSGGGGSGGGGSGGGGSDIQL |
| | | TQSPSSLSASVGDRVTITCRASQSISSYLNWYQQKPGKAPKL |
| | | LIYAASSLQSGVPSRFSGSGSGTDFTLTISSLQPEDFATYYCQ |
| | | QSYSTPRTFGQGTKVEIK |
| 195 | scFv variant 11 (V11) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRPD |
| | | DIVHVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |
| 196 | scFv variant 12 (V12) | MGWSCIILFLVATATGVHSEVQLVESGGGVVQPGRSLRLSC |
| | | AASGFPFSIYGMHWVRQAPGKGLEWVAVISYDGSHKYYAD |
| | | SVKGRFTISRDNSKNTLYLQMNSLRAEDTAVYYCAKEGRPD |
| | | DIVRVVAFDYWGQGTLVTVSSGGGGSGGGGSGGGGSDIQLT |
| | | QSPSSLSASVGDRVTITCRASQSISSYLNWYQQKPGKAPKLLI |
| | | YAASSLQSGVPSRFSGSGSGTDFTLTISSLQPEDFATYYCQQS |
| | | YSTPRTFGQGTKVEIK |

### Example 3. Identification of Synthetic Antibody Variants Based on Donor Antibody c002 Heavy Chain and Light Chain Mutations

Antibody variants based on mutations in the complementarity determining regions (CDRs) in heavy chain and light chain of donor antibody c002 were identified. In the table below, sixteen (16) heavy chains identified as v1_hc to v7_hc, v9_hc to v12_hc and dv1_hc to dv5_hc each correspond to an amino acid sequence of SEQ ID NO: 1 to SEQ ID NO: 16, respectively. One light chain dv1-lc has an amino acid sequence of SEQ ID NO: 17. An antibody comprising two light chains dv1-lc of an amino acid sequence of SEQ ID NO: 17 and two same heavy chains selected from any one of an amino acid sequence of SEQ ID NO: 1 to SEQ ID NO: 16 was identified.

| SEQ | Single chain | |
|---|---|---|
| ID NO | identifier | Protein Sequence |
| 1 | v1_hc | EVQLVESGGGVVQPGRSLRLSCAASGFPFSIFGMHWVRQAPGKGLE |
| | | WVAVISYDGSHKYYADSVKGRFTISRDNSKNTLYLQMNSLRAEDT |
| | | AVYYCAKEGRPDDIVHVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 2 | v2_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRGDDIVVVVAFDYWGQGTLVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 3 | v3_hc | EVQLVESGGGVVQPGRSLRLSCAASGFPFSIYGMHWVRQAPGKGL |
| | | EWVAVISYDGSNKYYADSVKGRFTISRDNSKNTLYLQMNSLRAED |
| | | TAVYYCAKEGRPDDIVRVVAFDYWGQGTLVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 4 | v4_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPSDIVHVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 5 | v5_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPSDIVGVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 6 | v6_hc | EVQLVESGGGVVQPGRSLRLSCAASGFPFSIFGMHWVRQAPGKGLE |
| | | WVAVISYDGSHKYYADSVKGRFTISRDNSKNTLYLQMNSLRAEDT |
| | | AVYYCAKEGRPSDIVHVVAFDYWGQGTLVTVSSASTKGPSVFPLAP |
| | | SSKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLN |
| | | GKEYKCKVSNKALPAPIEKTISKAKGQPREPQVYTLPPSREEMTKN |
| | | QVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 7 | v7_hc | EVQLVESGGGVVQPGRSLRLSCAASGFPFSIFGMHWVRQAPGKGLE |
| | | WVAVISYDGSHKYYADSVKGRFTISRDNSKNTLYLQMNSLRAEDT |
| | | AVYYCAKEGRPDDIVRVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 8 | v9_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRSSDIVLVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 9 | v10_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPSDIWHVSAFDYWGQGTLVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 10 | v11_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPDDIVHVVAFDYWGQGTLVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 11 | v12_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPDDIVRVVAFDYWGQGTLVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 12 | dv1_hc | EVQLVESGGGVVQPGRSLRLSCAASGFDYPISGMHWVRQAPGKGL |
| | | EWVAHISYDGSYKYYADSVKGRFTISRDNSKNTLYLQMNSLRAED |
| | | TAVYYCAKEGRPSDIVVVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 13 | dv2_hc | EVQLVESGGGVVQPGRSLRLSCAASGFDFPIAGMHWVRQAPGKGL |
| | | EWVAHISYDGSHKYYADSVKGRFTISRDNSKNTLYLQMNSLRAED |
| | | TAVYYCAKEGRPSDIVVVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 14 | dv3_hc | EVQLVESGGGVVQPGRSLRLSCAASGFDYPIYGMHWVRQAPGKGL |
| | | EWVAVISYDGSNKYYADSVKGRFTISRDNSKNTLYLQMNSLRAED |
| | | TAVYYCAKEGRPSDIVVVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 15 | dv4_hc | EVQLVESGGGVVQPGRSLRLSCAASGFPFSIYGMHWVRQAPGKGL |
| | | EWVAVISYDGSNKYYADSVKGRFTISRDNSKNTLYLQMNSLRAED |
| | | TAVYYCAKEGRPSDIVVVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 16 | dv5_hc | EVQLVESGGGVVQPGRSLRLSCAASGFPFSIYGMHWVRQAPGKGL |
| | | EWVAVISYDGSHKYYADSVKGRFTISRDNSKNTLYLQMNSLRAED |
| | | TAVYYCAKEGRPSDIVVVVAFDYWGQGTLVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 17 | dv1_lc | DIQLTQSPSSLSASVGDRVTITCRASQSISSYLNWYQQKPGKAPKLLI |
| | | YAASSLQSGVPSRFSGSGSGTDFTLTISSLQPEDFATYYCQQSHSLPR |
| | | TFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |

### Example 4. Identification of CDRs for Synthetic Antibody Variants Based on Donor Antibody c002 Heavy Chain and Light Chain Mutations

Complementarity determining regions (CDRs) in heavy chain and light chain of donor antibody c002 mutants were identified. In the table below, three CDRs (CDR-H1, CDR-H2, and CDR-H3) for sixteen (16) heavy chains identified for v1_hc to v7_hc, v9_hc to v12_hc and dv1_hc to dv5_hc each corresponding to an amino acid sequence of SEQ ID NO: 1 to SEQ ID NO: 16, respectively.

The one light chain dv1-lc having an amino acid sequence of SEQ ID NO: 17 was identified to have CDRs (CDR-L1, CDR-L2, and CDR-L3) as shown in the table below with the corresponding amino acid sequence numbers.

| SEQ ID | SEQ ID | Amino acid | SEQ ID | Amino acid | SEQ ID | Amino acid |
|---|---|---|---|---|---|---|
| NO for | NO for | sequence | NO for | sequence for | NO for | sequence for |
| full chain | CDR-L1 | for CDR-L1 | CDR-L2 | CDR-L2 | CDR-L3 | CDR-L3 |
| 17 | 140 | CRASQSISSYL | 151 | YAASSLQ | 162 | CQQSHSLPR |

The CDRs (CDR-H1, CDR-H2, and CDR-H3) for sixteen (16) heavy chains SEQ ID NO: 1 to SEQ ID NO: 16 are listed with the corresponding amino acid sequence numbers in the table below.

| SEQ ID | | | | | | Amino acid |
|---|---|---|---|---|---|---|
| NO for | SEQ ID | Amino acid | | Amino acid | SEQ ID | sequence |
| full | NO for | sequence for | SEQ ID NO | sequence for | NO for | for |
| chain | CDR-H1 | CDR-H1 | for CDR-H2 | CDR-H2 | CDR-H3 | CDR-H3 |
| 1 | 44 | IFGMH | 76 | VISYDGSHK | 108 | EGRPDDIVHV |
| | | | | YYADSVKG | | VAFDY |
| 2 | 45 | IYGMH | 77 | VISYDGSHK | 109 | EGRGDDIVVV |
| | | | | YYADSVKG | | VAFDY |
| 3 | 46 | IYGMH | 78 | VISYDGSNK | 110 | EGRPDDIVRV |
| | | | | YYADSVKG | | VAFDY |
| 4 | 47 | IYGMH | 79 | VISYDGSHK | 111 | EGRPSDIVHVV |
| | | | | YYADSVKG | | AFDY |
| 5 | 48 | IYGMH | 80 | VISYDGSHK | 112 | EGRPSDIVGVV |
| | | | | YYADSVKG | | AFDY |
| 6 | 49 | IFGMH | 81 | VISYDGSHK | 113 | EGRPSDIVHVV |
| | | | | YYADSVKG | | AFDY |
| 7 | 50 | IFGMH | 82 | VISYDGSHK | 114 | EGRPDDIVRV |
| | | | | YYADSVKG | | VAFDY |
| 8 | 51 | IYGMH | 83 | VISYDGSHK | 115 | EGRSSDIVLVV |
| | | | | YYADSVKG | | AFDY |
| 9 | 52 | IYGMH | 84 | VISYDGSHK | 116 | EGRPSDIWHVS |
| | | | | YYADSVKG | | AFDY |
| 10 | 53 | IYGMH | 85 | VISYDGSHK | 117 | EGRPDDIVHV |
| | | | | YYADSVKG | | VAFDY |
| 11 | 54 | IYGMH | 86 | VISYDGSHK | 118 | EGRPDDIVRV |
| | | | | YYADSVKG | | VAFDY |
| 12 | 55 | ISGMH | 87 | HISYDGSYK | 119 | EGRPSDIVVVV |
| | | | | YYADSVKG | | AFDY |
| 13 | 56 | IAGMH | 88 | HISYDGSHK | 120 | EGRPSDIVVVV |
| | | | | YYADSVKG | | AFDY |
| 14 | 57 | IYGMH | 89 | VISYDGSNK | 121 | EGRPSDIVVVV |
| | | | | YYADSVKG | | AFDY |
| 15 | 58 | IYGMH | 90 | VISYDGSNK | 122 | EGRPSDIVVVV |
| | | | | YYADSVKG | | AFDY |
| 16 | 59 | IYGMH | 91 | VISYDGSHK | 123 | EGRPSDIVVVV |
| | | | | YYADSVKG | | AFDY |

### Example 5. Identification of Synthetic Antibody Variants Based on Donor Antibody c118 Heavy Chain and Light Chain Mutations

Antibody variants based on mutations in the complementarity determining regions (CDRs) in heavy chain and light chain of donor antibody c118 were identified. In the table below, sixteen (16) heavy chains are listed, each identified as one of C118_v8 hc to C118_v22_hc, C118_a2 hc and C118_a6 hc corresponding to an amino acid sequence of SEQ ID NO: 18 to SEQ ID NO: 33, respectively.

| SEQ ID | single chain | |
|---|---|---|
| NO | identifier | Protein Sequence |
| 18 | C118_v8_hc | QVQLVESGGGVVQPGRSLRLSCAASGFEFSNYAMHWVRQAPGKGL |
| | | EWVAVINYDGSNKYYADSVKGRFTISRDNSKNTLYLQMNSLRAEDT |
| | | AIYYCASGYTGYDYFFHGEYFGLEVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 19 | C118_v9_hc | QVQLVESGGGVVQPGRSLRLSCAASGFHFSHYAMHWVRQAPGKGL |
| | | EWVAVINYDGSNKYYADSVKGRFTISRDNSKNTLYLQMNSLRAEDT |
| | | AIYYCASGYTGYDYFFGGEYFGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 20 | C118_v10_hc | QVQLVESGGGVVQPGRSLRLSCAASGFAFYNYAMHWVRQAPGKGL |
| | | EWVAVINYDGSNKYYADSVKGRFTISRDNSKNTLYLQMNSLRAEDT |
| | | AIYYCASGYTGYDYFFGGEYFGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 21 | C118_v12_hc | QVQLVESGGGVVQPGRSLRLSCAASGFAFNNYAMHWVRQAPGKGL |
| | | EWVAVINFDGSNKYYADSVKGRFTISRDNSKNTLYLQMNSLRAEDT |
| | | AIYYCASGYTGYDYFFGGDYFGLEVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 22 | C118_v13_hc | QVQLVESGGGVVQPGRSLRLSCAASGFKFSHYAMHWVRQAPGKGL |
| | | EWVAVINFDGSNKYYADSVKGRFTISRDNSKNTLYLQMNSLRAEDT |
| | | AIYYCASGYTGYDYFFNGEYYGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 23 | C118_v14_hc | QVQLVESGGGVVQPGRSLRLSCAASGFHFNNYAMHWVRQAPGKGL |
| | | EWVAVISYDGTNKYYADSVKGRFTISRDNSKNTLYLQMNSLRAEDT |
| | | AIYYCASGYTGYDYFFGGDYFGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 24 | C118_v15_hc | QVQLVESGGGVVQPGRSLRLSCAASGFTFNNYAMHWVRQAPGKGL |
| | | EWVAVISYDGSNKYYADSVKGRFTISRDNSKNTLYLQMNSLRAEDT |
| | | AIYYCASGYTGYDYFFGGDYYGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 25 | C118_v16_hc | QVQLVESGGGVVQPGRSLRLSCAASGFKFSNYAMHWVRQAPGKGL |
| | | EWVAVISYDGSNKYYADSVKGRFTISRDNSKNTLYLQMNSLRAEDT |
| | | AIYYCASGYTGYDYFFRGEYYGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 26 | C118_v17_hc | QVQLVESGGGVVQPGRSLRLSCAASGFHFSNYAMHWVRQAPGKGL |
| | | EWVAVINFDGSNKYYADSVKGRFTISRDNSKNTLYLQMNSLRAEDT |
| | | AIYYCASGYTGYDYFFSGEYFGLEVWGQGTTVTVSSASTKGPSVFPL |
| | | APSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 27 | C118_v18_hc | QVQLVESGGGVVQPGRSLRLSCAASGFHFNHYAMHWVRQAPGKGL |
| | | EWVAVINYDGTNKYYADSVKGRFTISRDNSKNTLYLQMNSLRAED |
| | | TAIYYCASGYTGYDYFFHGEYYGLDVWGQGTTVTVSSASTKGPSVF |
| | | PLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 28 | C118_v19_hc | QVQLVESGGGVVQPGRSLRLSCAASGFAFYNYAMHWVRQAPGKGL |
| | | EWVAVINYDGSNKYYADSVKGRFTISRDNSKNTLYLQMNSLRAEDT |
| | | AIYYCASGYTGYDYFVGGEYFGLEVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 29 | C118_v20_hc | QVQLVESGGGVVQPGRSLRLSCAASGFTFSNYAMHWVRQAPGKGL |
| | | EWVAVINFDGSNKYYADSVKGRFTISRDNSKNTLYLQMNSLRAEDT |
| | | AIYYCASGYTGYDYFVNGEYYGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 30 | C118_v21_hc | QVQLVESGGGVVQPGRSLRLSCAASGFAFYNYAMHWVRQAPGKGL |
| | | EWVAVINYDGTNKYYADSVKGRFTISRDNSKNTLYLQMNSLRAED |
| | | TAIYYCASGYTGYDYFFGGEYYGLEVWGQGTTVTVSSASTKGPSVF |
| | | PLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 31 | C118_v22_hc | QVQLVESGGGVVQPGRSLRLSCAASGFHFSHYAMHWVRQAPGKGL |
| | | EWVAVISFDGSNKYYADSVKGRFTISRDNSKNTLYLQMNSLRAEDT |
| | | AIYYCASGYTGYDYFFNGEYFGLDVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 32 | C118_a2_hc | QVQLVESGGGVVQPGRSLRLSCAASGFYFRNYAMHWVRQAPGKGL |
| | | EWVAVINYDGSNKYYADSVKGRFTISRDNSKNTLYLQMNSLRAEDT |
| | | AIYYCASGYTGYDYFVGGEYFGLEVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 33 | C118_a6_hc | QVQLVESGGGVVQPGRSLRLSCAASGFYFKNYAMHWVRQAPGKGL |
| | | EWVAVINYDGSNKYYADSVKGRFTISRDNSKNTLYLQMNSLRAEDT |
| | | AIYYCASGYTGYDYFVGGEYFGLEVWGQGTTVTVSSASTKGPSVFP |
| | | LAPSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLY |
| | | SKLTVDKSRWQQGNVFSCSVMHEALHNHYTQKSLSLSPGK |

In the table below, ten (10) light chains are listed, each identified as one of C118_v8_lc to C118_v10_lc, C118_v12_lc, C118_v14_lc, C118_v16_lc, C118_v21_lc, C118_a2_lc, C118_a6_lc and C118_a9_lc corresponding to an amino acid sequence of SEQ ID NO: 34 to SEQ ID NO: 43, respectively.

| SEQ ID | single chain | |
|---|---|---|
| NO | identifier | Protein Sequence |
| 34 | C118_v8_lc | QPVLTQSPSASASLGASVKLTCTLSSGHSSYAIAWHQQQPEKGPRYL |
| | | MKLNTDGSFNKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 35 | C118_v9_lc | QPVLTQSPSASASLGASVKLTCTLSSGHSSYAIAWHQQQPEKGPRYL |
| | | MKLNTDGSLEKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 36 | C118_v10_lc | QPVLTQSPSASASLGASVKLTCTLSSGHSSYAIAWHQQQPEKGPRYL |
| | | MKLNTDGSYEKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 37 | C118_v12_lc | QPVLTQSPSASASLGASVKLTCTLSSGHSSYAIAWHQQQPEKGPRYL |
| | | MKLNTDGSYDKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 38 | C118_v14_lc | QPVLTQSPSASASLGASVKLTCTLSSGHSSYAIAWHQQQPEKGPRYL |
| | | MKLNTDGSFEKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 39 | C118_v16_lc | QPVLTQSPSASASLGASVKLTCTLSSGHSSYAIAWHQQQPEKGPRYL |
| | | MKLNTDGSLDKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 40 | C118_v21_lc | QPVLTQSPSASASLGASVKLTCTLSSGHSSYAIAWHQQQPEKGPRYL |
| | | MKLNTDGSFDKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 41 | C118_a2_lc | QPVLTQSPSASASLGASVKLTCTLSSGHSSYAIAWHQQQPEKGPRYL |
| | | MKLATDGSYEMGHGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 42 | C118_a6_lc | QPVLTQSPSASASLGASVKLTCTLSSGHSSYAIAWHQQQPEKGPRYL |
| | | MKLNTDGSYEMGVGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |
| 43 | C118_a9_lc | QPVLTQSPSASASLGASVKLTCTLSSGHSSYAIAWHQQQPEKGPRYL |
| | | MKLNTDGSFEKGDGIPDRFSGSSSGAERYLTISSLQSEDEADYYCQT |
| | | WGTGILVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPTECS |

### Example 6. Identification of CDRs for Synthetic Antibody Variants Based on Donor Antibody c118 Heavy Chain Mutations

Complementarity determining regions (CDRs) in heavy chain and light chain of donor antibody c118 mutants were identified. In the table below, three CDRs (CDR-H1, CDR-H2, and CDR-H3) for sixteen (16) heavy chains identified for C118_v8_hc to C118_v22_hc, C118_a2_hc and C118_a6_hc each corresponding to an amino acid sequence of SEQ ID NO: 18 to SEQ ID NO: 33, respectively.

The CDRs (CDR-H1, CDR-H2, and CDR-H3) for sixteen (16) heavy chains SEQ ID NO: 18 to SEQ ID NO: 33 are listed with the corresponding amino acid sequence numbers in the table below.

| SEQ ID | SEQ ID | Amino acid | SEQ ID | Amino acid sequence | SEQ ID | Amino acid |
|---|---|---|---|---|---|---|
| NO for | NO for | sequence for | NO for | for CDR-H2 | NO for | sequence for |
| full chain | CDR-H1 | CDR-H1 | CDR-H2 | | CDR-H3 | CDR-H3 |
| 18 | 60 | NYAMH | 92 | VINYDGSNKYYADSV | 124 | GYTGYDYFF |
| | | | | KG | | HGEYFGLEV |
| 19 | 61 | HYAMH | 93 | VINYDGSNKYYADSV | 125 | GYTGYDYFF |
| | | | | KG | | GGEYFGLDV |
| 20 | 62 | NYAMH | 94 | VINYDGSNKYYADSV | 126 | GYTGYDYFF |
| | | | | KG | | GGEYFGLDV |
| 21 | 63 | NYAMH | 95 | VINFDGSNKYYADSV | 127 | GYTGYDYFF |
| | | | | KG | | GGDYFGLEV |
| 22 | 64 | HYAMH | 96 | VINFDGSNKYYADSV | 128 | GYTGYDYFF |
| | | | | KG | | NGEYYGLDV |
| 23 | 65 | NYAMH | 97 | VISYDGTNKYYADSV | 129 | GYTGYDYFF |
| | | | | KG | | GGDYFGLDV |
| 24 | 66 | NYAMH | 98 | VISYDGSNKYYADSV | 130 | GYTGYDYFF |
| | | | | KG | | GGDYYGLDV |
| 25 | 67 | NYAMH | 99 | VISYDGSNKYYADSV | 131 | GYTGYDYFF |
| | | | | KG | | RGEYYGLDV |
| 26 | 68 | NYAMH | 100 | VINFDGSNKYYADSV | 132 | GYTGYDYFFS |
| | | | | KG | | GEYFGLEV |
| 27 | 69 | HYAMH | 101 | VINYDGTNKYYADSV | 133 | GYTGYDYFF |
| | | | | KG | | HGEYYGLDV |
| 28 | 70 | NYAMH | 102 | VINYDGSNKYYADSV | 134 | GYTGYDYFV |
| | | | | KG | | GGEYFGLEV |
| 29 | 71 | NYAMH | 103 | VINFDGSNKYYADSV | 135 | GYTGYDYFV |
| | | | | KG | | NGEYYGLDV |
| 30 | 72 | NYAMH | 104 | VINYDGTNKYYADSV | 136 | GYTGYDYFF |
| | | | | KG | | GGEYYGLEV |
| 31 | 73 | HYAMH | 105 | VISFDGSNKYYADSV | 137 | GYTGYDYFF |
| | | | | KG | | NGEYFGLDV |
| 32 | 74 | NYAMH | 106 | VINYDGSNKYYADSV | 138 | GYTGYDYFV |
| | | | | KG | | GGEYFGLEV |
| 33 | 75 | NYAMH | 107 | VINYDGSNKYYADSV | 139 | GYTGYDYFV |
| | | | | KG | | GGEYFGLEV |

### Example 7. Identification of CDRs for Synthetic Antibody Variants Based on Donor Antibody c118 Light Chain Mutations

Complementarity determining regions (CDRs) in heavy chain and light chain of donor antibody c118 mutants were identified. In the table below, three CDRs (CDR-L1, CDR-L2, and CDR-L3) for each of the ten (10) light chains identified for C118_v8_lc to C118_v10_lc, C118_v12_lc, C118_v14_lc, C118_v16_lc, C118_v21_lc, C118_a2_lc, C118_a6_lc and C118_a9_lc corresponding to an amino acid sequence of SEQ ID NO: 34 to SEQ ID NO: 43, respectively.

The CDRs (CDR-L1, CDR-L2, and CDR-L3) for sixteen (10) light chains SEQ ID NO: 34 to SEQ ID NO: 43 are listed with the corresponding amino acid sequence numbers in the table below.

| SEQ ID | | | | | | |
|---|---|---|---|---|---|---|
| NO for | SEQ ID | Amino acid | SEQ ID | Amino acid | SEQ ID | Amino acid |
| full | NO for | sequence for | NO for | sequence | NO for | sequence for |
| chain | CDR-L1 | CDR-L1 | CDR-L2 | for CDR-L2 | CDR-L3 | CDR-L3 |
| 34 | 141 | TLSSGHSSYAIA | 152 | LNTDGSFNKGD | 163 | QTWGTGILV |
| 35 | 142 | TLSSGHSSYAIA | 153 | LNTDGSLEKGD | 164 | QTWGTGILV |
| 36 | 143 | TLSSGHSSYAIA | 154 | LNTDGSYEKGD | 165 | QTWGTGILV |
| 37 | 144 | TLSSGHSSYAIA | 155 | LNTDGSYDKGD | 166 | QTWGTGILV |
| 38 | 145 | TLSSGHSSYAIA | 156 | LNTDGSFEKGD | 167 | QTWGTGILV |
| 39 | 146 | TLSSGHSSYAIA | 157 | LNTDGSLDKGD | 168 | QTWGTGILV |
| 40 | 147 | TLSSGHSSYAIA | 158 | LNTDGSFDKGD | 169 | QTWGTGILV |
| 41 | 148 | TLSSGHSSYAIA | 159 | LATDGSYEMGH | 170 | QTWGTGILV |
| 42 | 149 | TLSSGHSSYAIA | 160 | LNTDGSYEMGV | 171 | QTWGTGILV |
| 43 | 150 | TLSSGHSSYAIA | 161 | LNTDGSFEKGD | 172 | QTWGTGILV |

### Identification of Synthetic Antibodies, Antibody Variants and Antibody Fragments

Synthetic antibodies, antibody variants and antibody fragments against SARS-CoV-2 were identified. Antibody variants 1 to 7 and 9-12 each comprise two light chains of L1 of SEQ ID NO 173 and two heavy chains selected from V1 to V7 and V9 to V12 of SEQ ID NOS. 174 to 184.

| SEQ | Antibody Variant | |
|---|---|---|
| ID NO | Full Chain | Protein Sequence |
| 173 | L1 - Light Chain | MGWSCIILFLVATATGVHSDIQLTQSPSSLSASVGDRVTITCRAS |
| | | QSISSYLNWYQQKPGKAPKLLIYAASSLQSGVPSRFSGSGSGTDF |
| | | TLTISSLQPEDFATYYCQQSYSTPRTFGQGTKVEIKRTVAAPSVFI |
| | | FPPSDEQLKSGTASVVCLLNNFYPREAKVQWKVDNALQSGNSQ |
| | | ESVTEQDSKDSTYSLSSTLTLSKADYEKHKVYACEVTHQGLSSP |
| | | VTKSFNRGEC |
| 174 | V1 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPDDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 175 | V2 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRGDDIVVVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 176 | V3 - Heavy Chain | MGWSCIILFLVATATGVHSEVQLVESGGGVVQPGRSLRLSCAAS |
| | | GFPFSIYGMHWVRQAPGKGLEWVAVISYDGSNKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 177 | V4 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRPSDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 178 | V5 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRPSDIVGVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 179 | V6 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPSDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 180 | V7 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 181 | V9 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRSSDIVLVVAF |
| | | DYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 182 | V10 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPSDIWHVSA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 183 | V11 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPDDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 184 | V12 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |

### Identification of Synthetic Antibodies, Antibody Variants and Antibody Fragments

Synthetic antibodies, antibody variants and antibody fragments against SARS-CoV-2 were identified. Antibody variants 1 to 7 and 9-12 each comprise two light chains of L1 of SEQ ID NO 173 and two heavy chains selected from V1 to V7 and V9 to V12 of SEQ ID NOS. 174 to 184.

| SEQ | Antibody Variant | |
|---|---|---|
| ID NO | Full Chain | Protein Sequence |
| 173 | L1 - Light Chain | MGWSCIILFLATATGVHSDIQLTQSPSSLSASVGDRVTITCRAS |
| | | QSISSYLNWYQQKPGKAPKLLIYAASSLQSGVPSRFSGSGSGTDF |
| | | TLTISSLQPEDFATYYCQQSYSTPRTFGQGTKVEIKRTVAAPSVFI |
| | | FPPSDEQLKSGTASVVCLLNNFYPREAKVQWKVDNALQSGNSQ |
| | | ESVTEQDSKDSTYSLSSTLTLSKADYEKHKVYACEVTHQGLSSP |
| | | VTKSFNRGEC |
| 174 | V1 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPDDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 175 | V2 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRGDDIVVVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 176 | V3 - Heavy Chain | MGWSCIILFLVATATGVHSEVQLVESGGGVVQPGRSLRLSCAAS |
| | | GFPFSIYGMHWVRQAPGKGLEWVAVISYDGSNKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 177 | V4 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRPSDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 178 | V5 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRPSDIVGVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 179 | V6 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPSDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 180 | V7 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 181 | V9 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGR |
| | | FTISRDNSKNTLYLQMNSLRAEDTAVYYCAKEGRSSDIVLVVAF |
| | | DYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 182 | V10 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPSDIWHVSA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 183 | V11 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPDDIVHVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 184 | V12 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGR |
| | | FTISRDNSKNTLYLQMNSLRAEDTAVYYCAKEGRPDDIVRVVA |
| | | FDYWGQGTLVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKRVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |

### Identification of Synthetic Antibodies, Antibody Variants and Antibody Fragments

Synthetic antibodies, antibody variants and antibody fragments against SARS-CoV-2 were identified. Antibody variants 1 to 7 and 9-12 each comprise two light chains of L1 of SEQ ID NO 173 and two heavy chains selected from V1 to V7 and V9 to V12 of SEQ ID NOS. 174 to 184.

| SEQ | Antibody Variant | |
|---|---|---|
| ID NO | Full Chain | Protein Sequence |
| 173 | L1 - Light Chain | MGWSCIILFLVATATGVHSDIQLTQSPSSLSASVGDRVTITCRAS |
| | | QSISSYLNWYQQKPGKAPKLLIYAASSLQSGVPSRFSGSGSGTDF |
| | | TLTISSLQPEDFATYYCQQSYSTPRTFGQGTKVEIKRTVAAPSVFI |
| | | FPPSDEQLKSGTASVVCLLNNFYPREAKVQWKVDNALQSGNSQ |
| | | ESVTEQDSKDSTYSLSSTLTLSKADYEKHKVYACEVTHQGLSSP |
| | | VTKSFNRGEC |
| 174 | V1 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGRF |
| | | TISRDNSKNTLYLQMNSLRAEDTAVYYCAKEGRPDDIVHVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 175 | V2 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGRF |
| | | TISRDNSKNTLYLQMNSLRAEDTAVYYCAKEGRGDDIVVVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 176 | V3 - Heavy Chain | MGWSCIILFLVATATGVHSEVQLVESGGGVVQPGRSLRLSCAAS |
| | | GFPFSIYGMHWVRQAPGKGLEWVAVISYDGSNKYYADSVKGRF |
| | | TISRDNSKNTLYLQMNSLRAEDTAVYYCAKEGRPDDIVRVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 177 | V4 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGRF |
| | | TISRDNSKNTLYLQMNSLRAEDTAVYYCAKEGRPSDIVHVVAFD |
| | | YWGQGTLVTVSSASTKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 178 | V5 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGRF |
| | | TISRDNSKNTLYLQMNSLRAEDTAVYYCAKEGRPSDIVGVVAFD |
| | | YWGQGTLVTVSSASTKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 179 | V6 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGRF |
| | | TISRDNSKNTLYLQMNSLRAEDTAVYYCAKEGRPSDIVHVVAFD |
| | | YWGQGTLVTVSSASTKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 180 | V7 - Heavy Chain | MGWSCIILFLVATATGVHSEVQLVESGGGVVQPGRSLRLSCAAS |
| | | GFPFSIFGMHWVRQAPGKGLEWVAVISYDGSHKYYADSVKGRF |
| | | TISRDNSKNTLYLQMNSLRAEDTAVYYCAKEGRPDDIVRVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 181 | V9 - Heavy Chain | MGWSCIILFLVATATGVHSEVQLVESGGGVVQPGRSLRLSCAAS |
| | | GFPFSIYGMHWVRQAPGKGLEWVAVISYDGSHKYYADSVKGRF |
| | | TISRDNSKNTLYLQMNSLRAEDTAVYYCAKEGRSSDIVLVVAFD |
| | | YWGQGTLVTVSSASTKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 182 | V10 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGRF |
| | | TISRDNSKNTLYLQMNSLRAEDTAVYYCAKEGRPSDIWHVSAFD |
| | | YWGQGTLVTVSSASTKGPSVFPLAPSSKSTSGGTAALGCLVKDY |
| | | FPEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 183 | V11 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGRF |
| | | TISRDNSKNTLYLQMNSLRAEDTAVYYCAKEGRPDDIVHVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 184 | V12 - Heavy | MGWSCIILFLVATATGVHSEVQLVESGGGVVQPGRSLRLSCAAS |
| | Chain | GFPFSIYGMHWVRQAPGKGLEWVAVISYDGSHKYYADSVKGRF |
| | | TISRDNSKNTLYLQMNSLRAEDTAVYYCAKEGRPDDIVRVVAF |
| | | DYWGQGTLVTVSSASTKGPSVFPLAPSSKSTSGGTAALGCLVKD |
| | | YFPEPVTVSWNSGALTSGVHTFPAVLQSSGLYSLSSVVTVPSSSL |
| | | GTQTYICNVNHKPSNTKVDKRVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |

### Example 8. Neutralization Assays

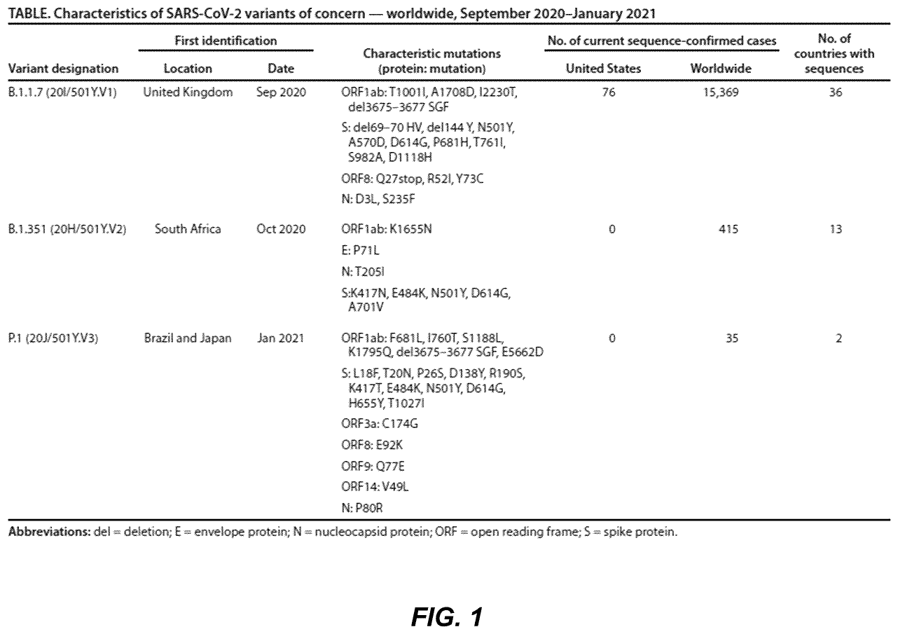

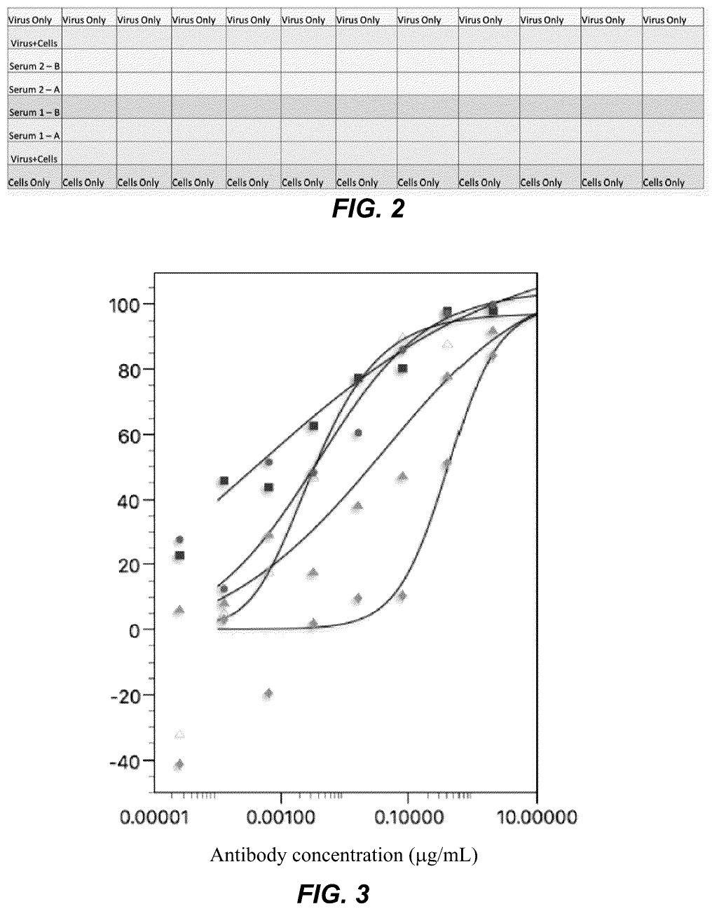

SARS2 pseudovirus neutralization by the antibody variants and fragments as described herein was evaluated using a 96-well assay format of an in-house poly-L-Lys coated plate free of polybrene as shown in FIG. 2. For example, EC50 for pseudovirus for SARS-CoV-2 501Y.V2 or SARS2-CoV-2 B1.1.7 as indicated in FIG. 1 were measured and data was read robotically.

As shown in FIG. 3, SARS2 pseudovirus was neutralized at with antibody variants at different concentrations, including C002 V1 (a heavy chain amino acid sequence of SEQ ID NO: 174 and a light chain amino acid sequence of SEQ ID NO: 173) (solid circle, IC50 0.0035 μg/mL), C002 V2 (a heavy chain amino acid sequence of SEQ ID NO: 175 and a light chain amino acid sequence of SEQ ID NO: 173) (solid square, IC50 0.0004 μg/mL), C002 V3(a heavy chain amino acid sequence of SEQ ID NO: 176 and a light chain amino acid sequence of SEQ ID NO: 173) (solid triangle, IC50 0.0024 μg/mL), C002 V4 (a heavy chain amino acid sequence of SEQ ID NO: 177 and a light chain amino acid sequence of SEQ ID NO: 173) (solid diamond, IC50 0.40 μg/mL) and C002 V5 (a heavy chain amino acid sequence of SEQ ID NO: 178 and a light chain amino acid sequence of SEQ ID NO: 173) (blank triangle, IC50 0.0033 μg/mL).

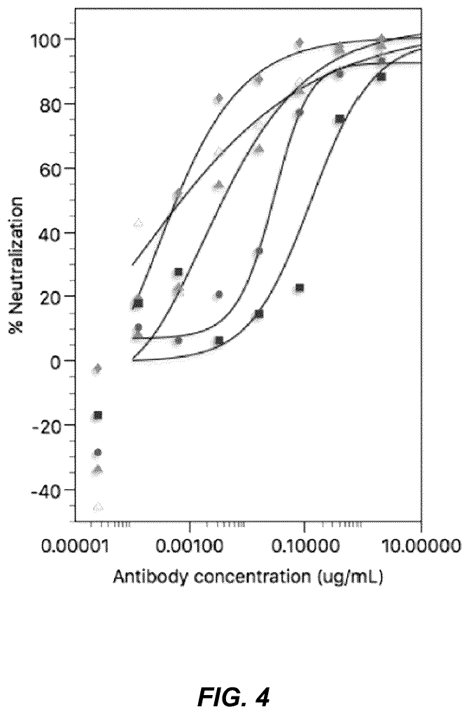

As shown in FIG. 4, SARS2 pseudovirus was assayed with antibody variants at different antibody variant concentrations, including C002 V2 (a heavy chain amino acid sequence of SEQ ID NO: 175 and a light chain amino acid sequence of SEQ ID NO: 173) (solid circle, IC50 0.028 μg/mL), C002 V7 (a heavy chain amino acid sequence of SEQ ID NO: 180 and a light chain amino acid sequence of SEQ ID NO: 173) (solid square, IC50 0.13 μg/mL), C002 V9 (a heavy chain amino acid sequence of SEQ ID NO: 181 and a light chain amino acid sequence of SEQ ID NO: 173) (solid triangle, IC50 0.0036 μg/mL), C002 V10 (a heavy chain amino acid sequence of SEQ ID NO: 182 and a light chain amino acid sequence of SEQ ID NO: 173) (solid diamond, IC50 0.001 μg/mL) and C005 IgG (blank triangle, IC50 0.001 μg/mL).

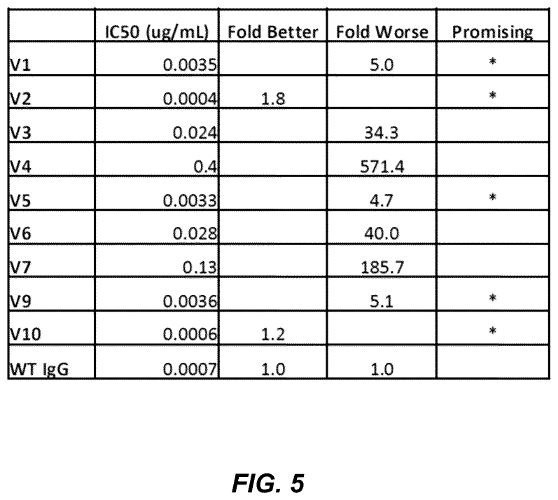

The neutralization assay results are presented in FIG. 5 which shows a ratio of IC50 for each antibody variant to WT IgG for V1, V3, V4, V5, V6, V7 and V9 and a ratio of IC50 for WT IgG to V2 and V10.

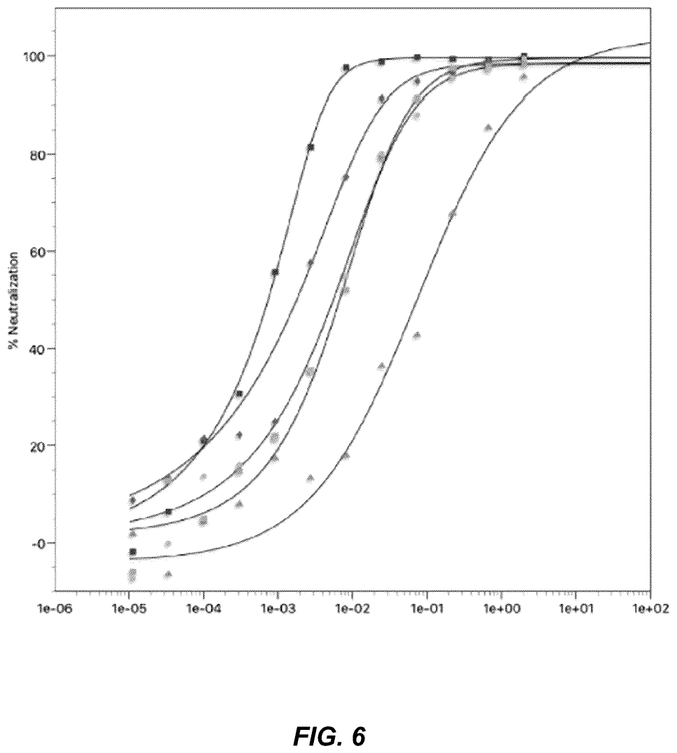

As shown in FIG. 6, SARS2-CoV-2 D614G pseudovirus was assayed with antibody variants at different antibody concentrations, including C002 V1 (a heavy chain amino acid sequence of SEQ ID NO: 174 and a light chain amino acid sequence of SEQ ID NO: 173) (lower solid square, IC50 6.5 vg/mL), C002 V2 (a heavy chain amino acid sequence of SEQ ID NO: 175 and a light chain amino acid sequence of SEQ ID NO: 173) (solid triangle, IC50 73 ng/mL), C002 V3 (a heavy chain amino acid sequence of SEQ ID NO: 176 and a light chain amino acid sequence of SEQ ID NO: 173) (solid diamond, IC50 1.8 ng/mL), C002 V4 (a heavy chain amino acid sequence of SEQ ID NO: 177 and a light chain amino acid sequence of SEQ ID NO: 173) (upper solid square, IC50 0.7 ng/mL) and C002 V5(a heavy chain amino acid sequence of SEQ ID NO: 178 and a light chain amino acid sequence of SEQ ID NO: 173) (solid circle, IC50 5.5 ng/mL), 1:6 virus dilution (new stock of virus).

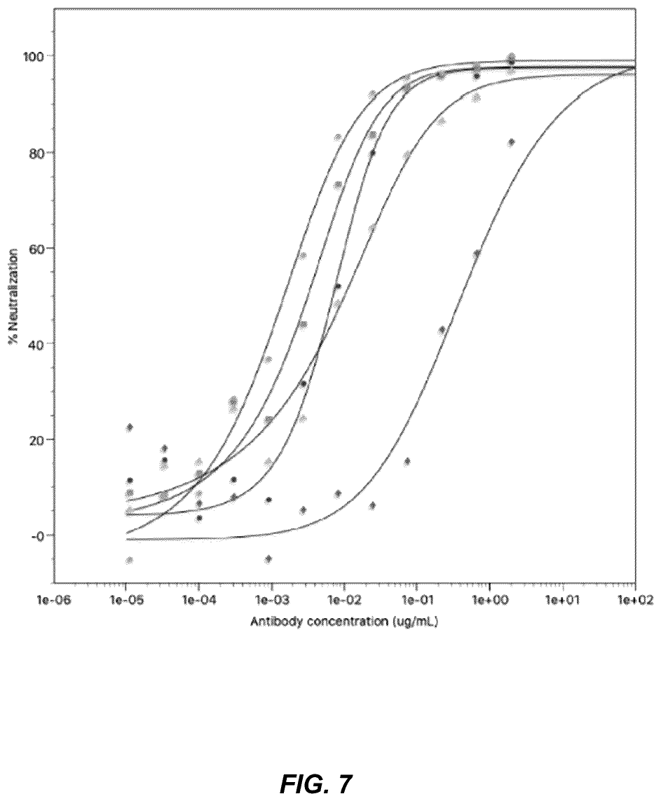

Also as shown in FIG. 7, SARS2-CoV-2 D614G pseudovirus was assayed with antibody variants at different antibody concentrations, including C002 V6 (a heavy chain amino acid sequence of SEQ ID NO: 179 and a light chain amino acid sequence of SEQ ID NO: 173) (upper solid circle, IC50 1.4 ng/mL), C002 V7 (a heavy chain amino acid sequence of SEQ ID NO: 180 and a light chain amino acid sequence of SEQ ID NO: 173) (solid square, IC50 3.2 ng/mL), C002 V9 (a heavy chain amino acid sequence of SEQ ID NO: 181 and a light chain amino acid sequence of SEQ ID NO: 173) (solid triangle, IC50 10 ng/mL), C002 V10 (a heavy chain amino acid sequence of SEQ ID NO: 182 and a light chain amino acid sequence of SEQ ID NO: 173) (solid diamond, IC50 410 ng/mL) and C002 IgG (lower solid circle, IC50 7.2 ng/mL), 1:6 virus dilution (new stock of virus).

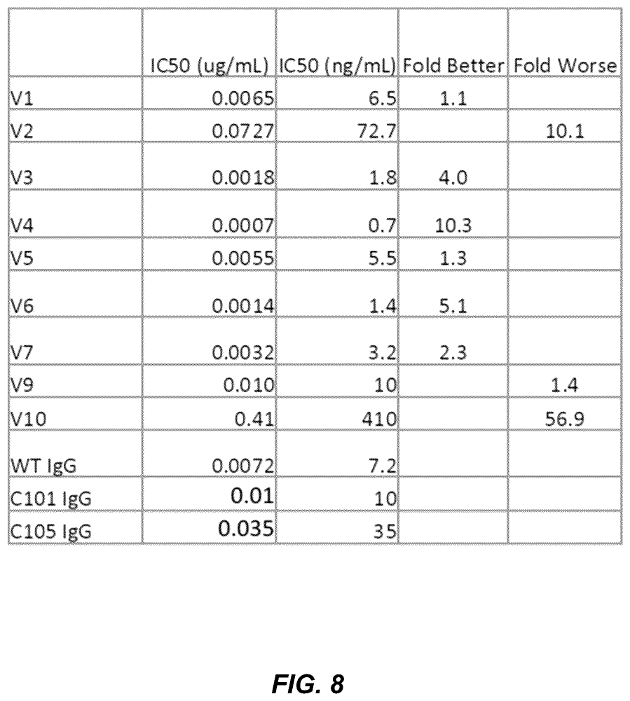

The results in FIG. 6 and FIG. 7 of SARS2-CoV-2 D614G pseudovirus assay are presented in FIG. 8 which shows a ratio of IC50 for each antibody variant to WT IgG for V2, V9 and V10 and a ratio of IC50 for WT IgG to V1, V3, V4, V5, V6, and V7. Included in the Table are IC50 for C101 IgG and C105 IgG. IC 50 for C002 IgG, C101 IgG and C105 IgG are reported in literature to be 8.9 ng/mL, 8.2 ng/mL, and 26 ng/mL respectively. The assay data were analyzed by a 5-parameter fit.

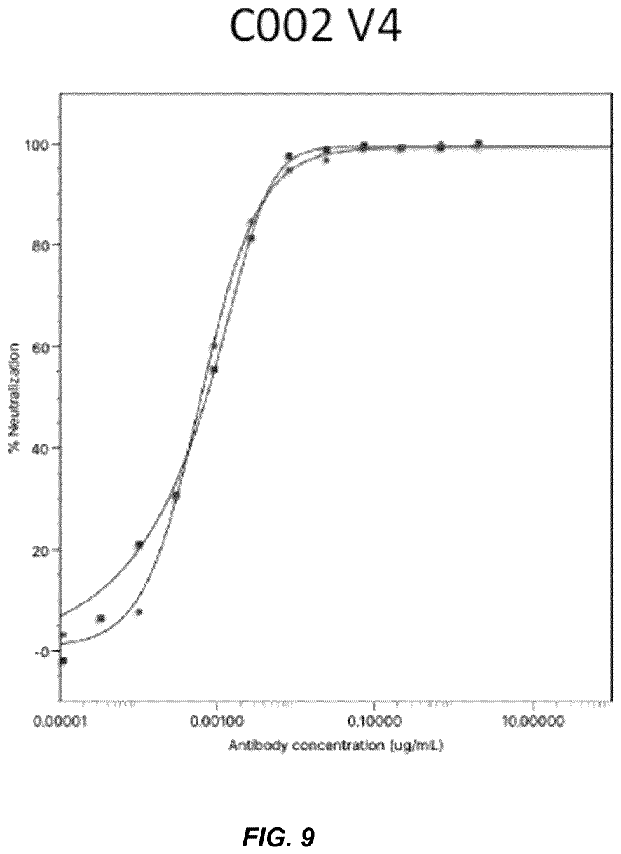

As shown in FIG. 9, SARS2 pseudovirus was assayed with antibody variant at different antibody concentrations for C002 V4 (a heavy chain amino acid sequence of SEQ ID NO: 177 and a light chain amino acid sequence of SEQ ID NO: 173) (solid circle, assay of Dec. 11, 2020; and solid square, assay of Dec. 17, 2020).

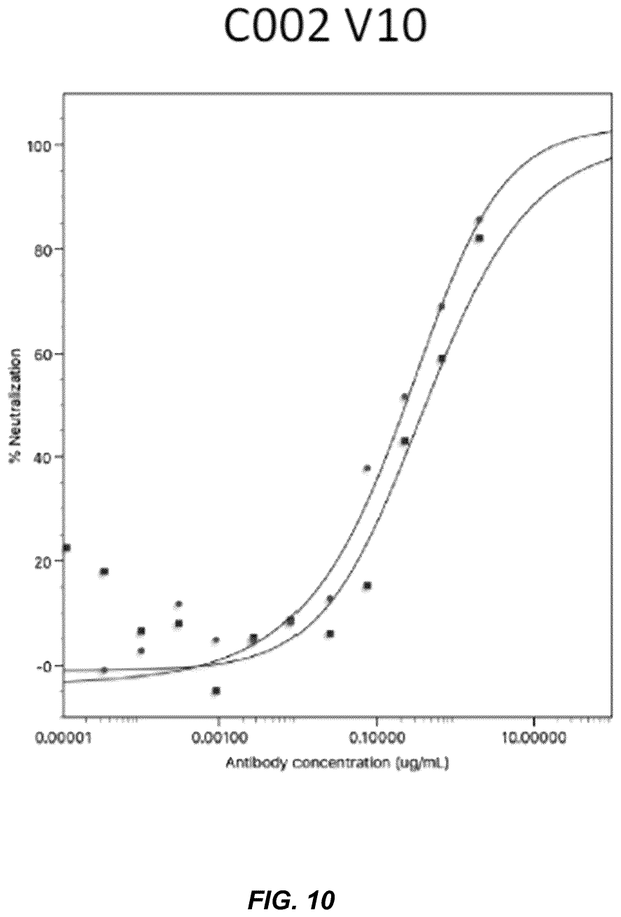

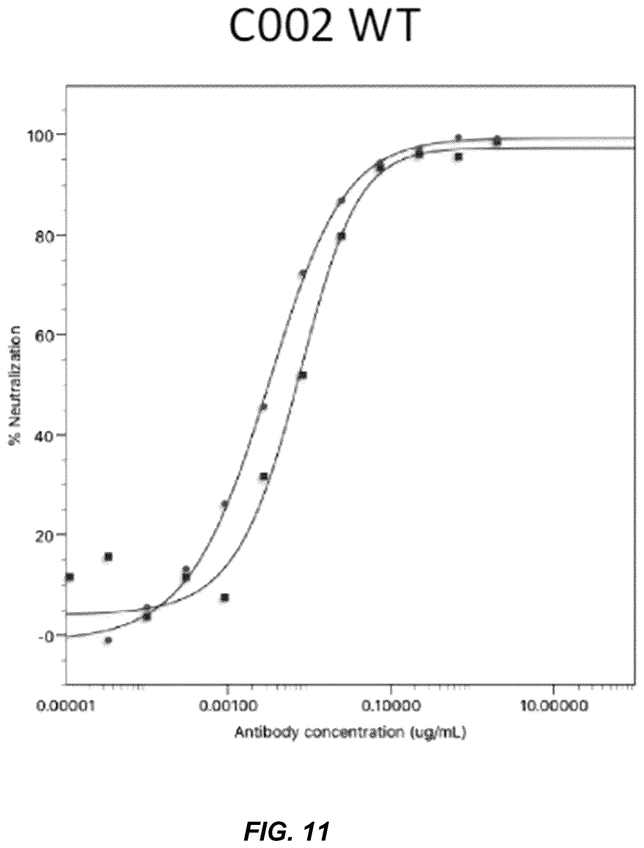

As shown in FIG. 10, SARS2 pseudovirus was assayed with antibody variant at different antibody concentrations for C002 V10 (a heavy chain amino acid sequence of SEQ ID NO: 182 and a light chain amino acid sequence of SEQ ID NO: 173) (solid circle, assay of Dec. 11, 2020; and solid square, assay of Dec. 17, 2020). In a similar manner, FIG. 11 shows SARS2 pseudovirus assay with antibody C002 WT at different antibody concentrations for C002 WT (solid circle, assay of Dec. 11, 2020; and solid square, assay of Dec. 17, 2020).

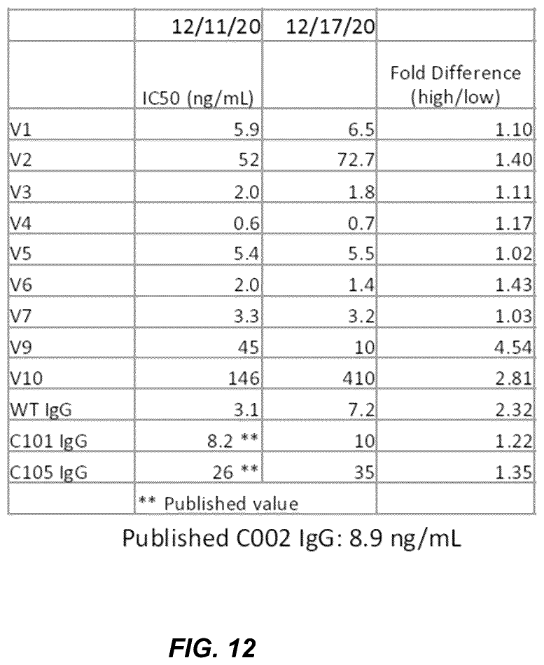

FIG. 12 shows duplicate results of SARS2 pseudovirus assay for c002 V1, V2, V3, V4, V5, V6, V7, V9 and V10 on two separate days and presenting IC50 in ng/mL and a ratio of IC50 for each antibody variant to WT IgG for c002 V1, V2, V3, V4, V5, V6, V7, V9 and V10.

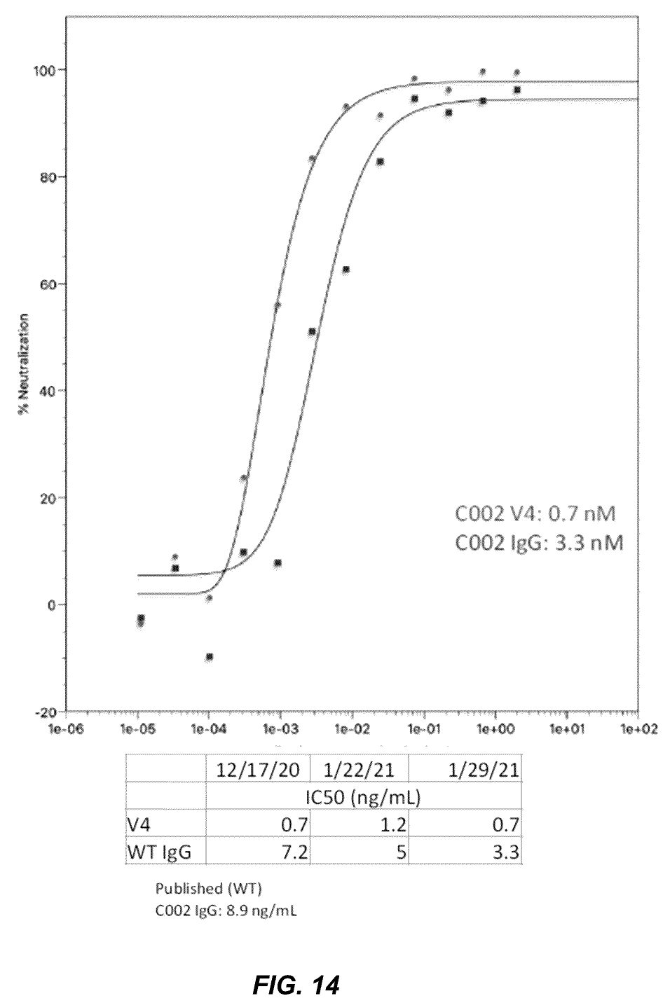

Results of SARS2 pseudovirus assay with antibody variant C002 V4 was shown in FIG. 13 for C002 V4 (solid circle) and C002 IgG (solid square). The table in FIG. 14 shows triplicate results of SARS2 pseudovirus assay for c002 V4 and WT IgG on three separate days and presenting corresponding IC50 data in ng/mL.

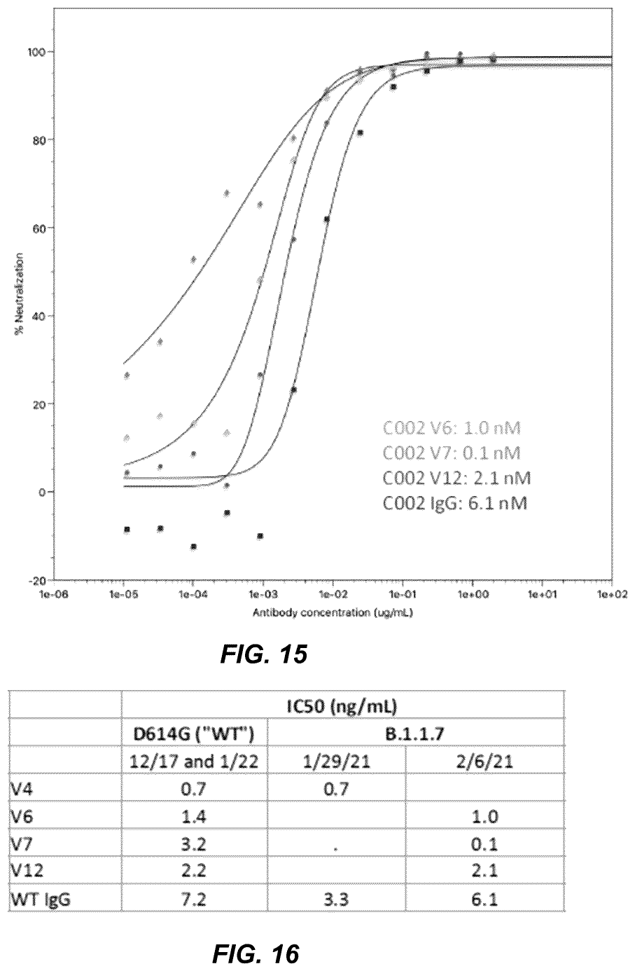

SARS2-CoV-2 UK isolate (B.1.1.7) pseudovirus assay with antibody variants at different antibody concentrations was shown in FIG. 15 for C002 V6 (solid triangle, IC50 1.0 ng/mL), C002 V7 (solid diamond, IC50 0.1 ng/mL), C002 V12 (a heavy chain amino acid sequence of SEQ ID NO: 184 and a light chain amino acid sequence of SEQ ID NO: 173) (solid circle, IC50 2.1 ng/mL), and C002 IgG (solid square, IC50 6.1 ng/mL), 1:6 virus dilution (new stock of virus).

FIG. 16 shows triplicate results of SARS2-CoV-2 UK isolate (B.1.1.7) pseudovirus assay for c002 V4, V6, V7, V12, and WT IgG on different days and presenting IC50 in ng/mL for each antibody variant and WT IgG. As indicated in FIG. 1, the mutations in UK isolate include H69-70V deletion, Y144 deletion, N501Y, A570D, D614G, P681H, T716I, S982A and D1118H which correspond to positions in the c002 epitopes.

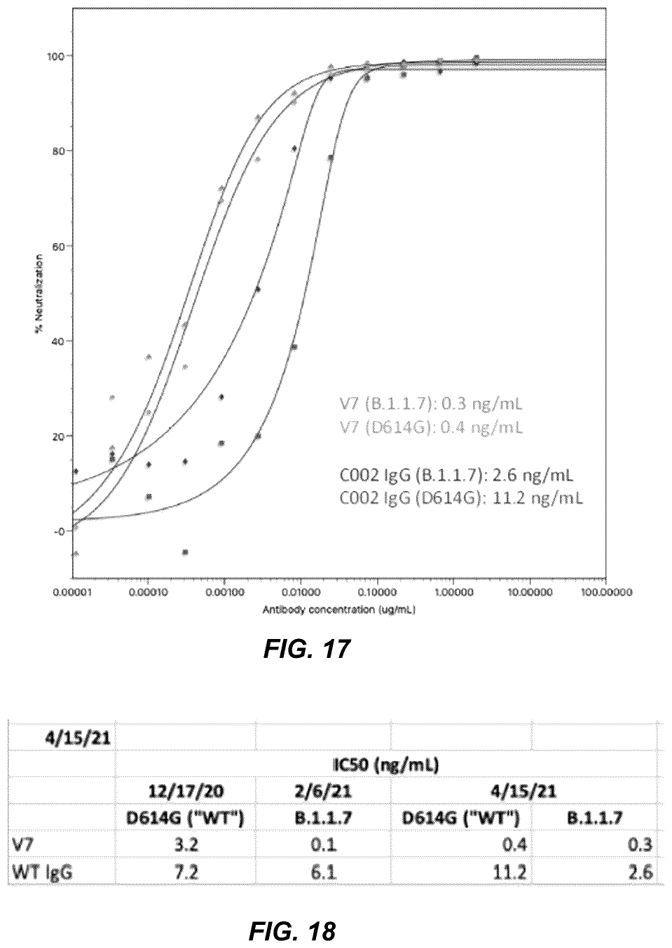

SARS2-CoV-2 UK isolate (B.1.1.7) and D614G (“WT”) pseudovirus assay with antibody variants was shown in FIG. 17 for C002 V7 (B.1.1.7) (solid triangle, IC50 0.3 ng/mL), C002 V7 (D614G) (solid circle, IC50 0.4 ng/mL), and C002 IgG (B.1.1.7) (solid diamond, IC50 2.6 ng/mL), C002 IgG (D614G) (solid square, IC50 11.2 ng/mL). As indicated in FIG. 1, the mutations in UK isolate include H69-70V deletion, Y144 deletion, N501Y, A570D, D614G, P681H, T716I, S982A and D1118H which correspond to positions in the c002 epitopes. IC 50 for C002 IgG against WT is reported in literature to be 8.9 ng/mL.

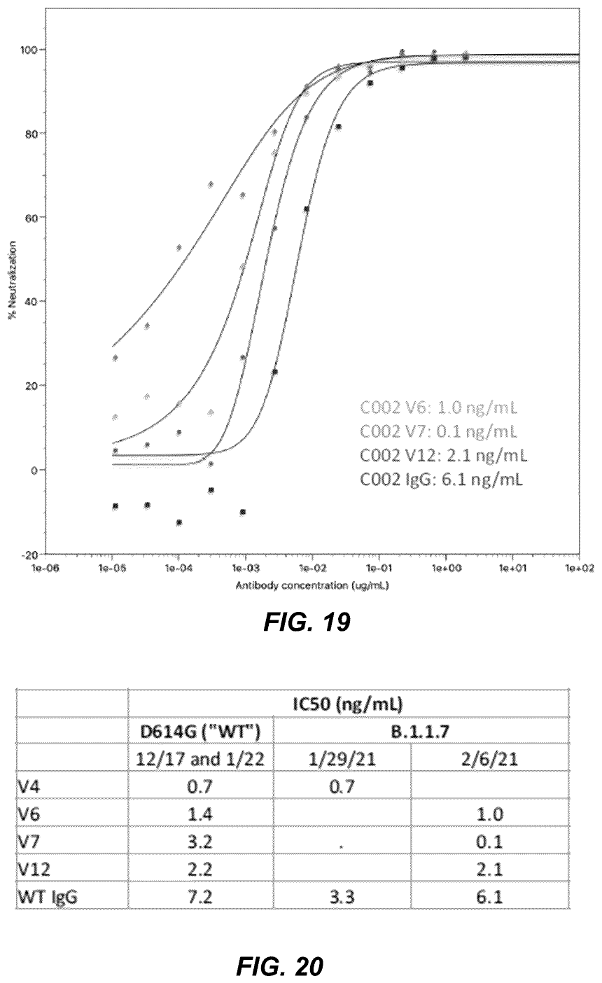

The table in FIG. 18 shows duplicate results of SARS2-CoV-2 UK isolate (B.1.1.7) and D614G (“WT”) pseudovirus assay for V7, and WT IgG on different days and presenting IC50 in ng/mL for c002 V7 antibody variant and WT IgG. SARS2-CoV-2 UK isolate (B.1.1.7) pseudovirus assay with antibody variants was shown in FIG. 19 depicts with results of percentage neutralization vs. antibody concentrations for C002 V6 (solid triangle, IC50 1.0 ng/mL), C002 V7 (solid diamond, IC50 0.1 ng/mL), and C002 V12 (solid circle, IC50 2.1 ng/mL), and C002 IgG (solid square, IC50 6.1 ng/mL). As indicated in FIG. 1, the mutations in UK isolate include H69-70V deletion, Y144 deletion, N501Y, A570D, D614G, P681H, T716I, S982A and D1118H which correspond to positions in the c002 epitopes. IC 50 for C002 IgG against WT is reported in literature to be 8.9 ng/mL. FIG. 20 shows results of SARS2-CoV-2 UK isolate (B.1.1.7) and D614G (“WT”) pseudovirus assay for c002 V4, V6, V7, V12, and WT IgG on different days and presenting IC50 in ng/mL for c002 V4, V6, V7, V12, and antibody variants and c002 WT IgG, respectively.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An antibody fragment, wherein the fragment has specificity to a sarbecovirus and comprises:
a heavy chain variable region (VH) comprising
(a) a CDR-H1 comprising an amino acid sequence selected from the group consisting of SEQ ID NOs: 44-75 or a variant thereof having a single substitution, deletion or insertion from any one of SEQ ID NOs: 44-75;
(b) a CDR-H2 comprising an amino acid sequence selected from the group consisting of SEQ ID NOs: 76-107 or a variant thereof having a single substitution, deletion or insertion from any one of SEQ ID NOs: 76-107;
(c) a CDR-H3 comprising an amino acid sequence selected from the group consisting of SEQ ID NOs: 108-139 or a variant thereof having a single substitution, deletion or insertion from any one of SEQ ID NOs: 108-139; and/or
a light chain variable region (VL) comprising
(a) a CDR-L1 comprising an amino acid sequence selected from the group consisting of SEQ ID NOs: 140-150 or a variant thereof having a single substitution, deletion or insertion from any one of SEQ ID NOs: 140-150;
(b) a CDR-L2 comprising an amino acid sequence selected from the group consisting of SEQ ID NOs: 151-161 or a variant thereof having a single substitution, deletion or insertion from any one of SEQ ID NOs: 151-161; and
(c) a CDR-L3 comprising an amino acid sequence selected from the group consisting of SEQ ID NOs: 162-172 or a variant thereof having a single substitution, deletion or insertion from any one of SEQ ID NOs: 162-172.

2. The antibody fragment of claim 1, comprising:
(a) the CDR-H1 comprising the amino acid sequence of SEQ ID NO: 44,
(b) the CDR-H2 comprising the amino acid sequence of SEQ ID NO: 76, and
(c) the CDR-H3 comprising the amino acid sequence of SEQ ID NO: 108;
or
(a) the CDR-H1 comprising the amino acid sequence of SEQ ID NO: 45,
(b) the CDR-H2 comprising the amino acid sequence of SEQ ID NO: 77, and
(c) the CDR-H3 comprising the amino acid sequence of SEQ ID NO: 109;
or
(a) the CDR-H1 comprising the amino acid sequence of SEQ ID NO: 46,
(b) the CDR-H2 comprising the amino acid sequence of SEQ ID NO: 78, and
(c) the CDR-H3 comprising the amino acid sequence of SEQ ID NO: 110;
or
(a) the CDR-H1 comprising the amino acid sequence of SEQ ID NO: 47,
(b) the CDR-H2 comprising the amino acid sequence of SEQ ID NO: 79, and
(c) the CDR-H3 comprising the amino acid sequence of SEQ ID NO: 111;
or
(a) the CDR-H1 comprising the amino acid sequence of SEQ ID NO: 48,
(b) the CDR-H2 comprising the amino acid sequence of SEQ ID NO: 80, and
(c) the CDR-H3 comprising the amino acid sequence of SEQ ID NO: 112;
or
(a) the CDR-H1 comprising the amino acid sequence of SEQ ID NO: 49,
(b) the CDR-H2 comprising the amino acid sequence of SEQ ID NO: 81, and
(c) the CDR-H3 comprising the amino acid sequence of SEQ ID NO: 113;
or
(a) the CDR-H1 comprising the amino acid sequence of SEQ ID NO: 50,
(b) the CDR-H2 comprising the amino acid sequence of SEQ ID NO: 82, and
(c) the CDR-H3 comprising the amino acid sequence of SEQ ID NO: 114;
or
(a) the CDR-H1 comprising the amino acid sequence of SEQ ID NO: 51,
(b) the CDR-H2 comprising the amino acid sequence of SEQ ID NO: 83, and
(c) the CDR-H3 comprising the amino acid sequence of SEQ ID NO: 115;
or
(a) the CDR-H1 comprising the amino acid sequence of SEQ ID NO: 52,
(b) a CDR-H2 comprising an amino acid sequence of SEQ ID NO: 84, and
(c) the CDR-H3 comprising the amino acid sequence of SEQ ID NO: 116;
or
(a) the CDR-H1 comprising the amino acid sequence of SEQ ID NO: 53,
(b) the CDR-H2 comprising the amino acid sequence of SEQ ID NO: 85, and
(c) the CDR-H3 comprising the amino acid sequence of SEQ ID NO: 117;
or
(a) the CDR-H1 comprising the amino acid sequence of SEQ ID NO: 54,
(b) the CDR-H2 comprising the amino acid sequence of SEQ ID NO: 86, and
(c) the CDR-H3 comprising the amino acid sequence of SEQ ID NO: 118;
or
(a) the CDR-H1 comprising an amino acid sequence of SEQ ID NO: 55,
(b) a CDR-H2 comprising the amino acid sequence of SEQ ID NO: 87, and
(c) the CDR-H3 comprising the amino acid sequence of SEQ ID NO: 119;
or
(a) the CDR-H1 comprising the amino acid sequence of SEQ ID NO: 56,
(b) the CDR-H2 comprising the amino acid sequence of SEQ ID NO: 88, and
(c) the CDR-H3 comprising the n amino acid sequence of SEQ ID NO: 120;
or
(a) the CDR-H1 comprising the amino acid sequence of SEQ ID NO: 57,
(b) the CDR-H2 comprising the amino acid sequence of SEQ ID NO: 89, and
(c) the CDR-H3 comprising the amino acid sequence of SEQ ID NO: 121;
or
(a) the CDR-H1 comprising the amino acid sequence of SEQ ID NO: 58,
(b) the CDR-H2 comprising the amino acid sequence of SEQ ID NO: 90, and
(c) the CDR-H3 comprising the amino acid sequence of SEQ ID NO: 122;
or
(a) the CDR-H1 comprising the amino acid sequence of SEQ ID NO: 59,
(b) the CDR-H2 comprising the amino acid sequence of SEQ ID NO: 91, and
(c) the CDR-H3 comprising the amino acid sequence of SEQ ID NO: 123.

3.-19. (canceled)

20. The antibody fragment of claim 1, comprising:
(a) the CDR-L1 comprising the amino acid sequence of SEQ ID NO: 140;
(b) the CDR-L2 comprising the amino acid sequence of SEQ ID NO: 151; and
(c) the CDR-L3 comprising the amino acid sequence of SEQ ID NO: 162.

21. An antibody or a fragment thereof, wherein the antibody or the fragment thereof comprises at least a heavy chain variable region and at least a light chain variable region of claim 1.

22.-24. (canceled)

25. The antibody fragment of claim 1, comprising:
(a) the CDR-H1 comprising the amino acid sequence of SEQ ID NO: 60.
(b) the CDR-H2 comprising the amino acid sequence of SEQ ID NO: 92, and
(c) the CDR-H3 comprising the amino acid sequence of SEQ ID NO: 124;
or
(a) the CDR-H1 comprising the amino acid sequence of SEQ ID NO: 61,
(b) the CDR-H2 comprising the amino acid sequence of SEQ ID NO: 93, and
(c) the CDR-H3 comprising the amino acid sequence of SEQ ID NO: 125;
or
(a) the CDR-H1 comprising the amino acid sequence of SEQ ID NO: 62,
(b) the CDR-H2 comprising the amino acid sequence of SEQ ID NO: 94, and
(c) the CDR-H3 comprising the amino acid sequence of SEQ ID NO: 126;
or
(a) the CDR-H1 comprising the amino acid sequence of SEQ ID NO: 63,
(b) the CDR-H2 comprising the amino acid sequence of SEQ ID NO: 95, and
(c) the CDR-H3 comprising the amino acid sequence of SEQ ID NO: 127;
or
(a) the CDR-H1 comprising the amino acid sequence of SEQ ID NO: 64,
(b) the CDR-H2 comprising the amino acid sequence of SEQ ID NO: 96, and
(c) the CDR-H3 comprising the amino acid sequence of SEQ ID NO: 128;
or
(a) the CDR-H1 comprising the amino acid sequence of SEQ ID NO: 65,
(b) the CDR-H2 comprising the amino acid sequence of SEQ ID NO: 97, and
(c) the CDR-H3 comprising the amino acid sequence of SEQ ID NO: 129;
or
(a) the CDR-H1 comprising the amino acid sequence of SEQ ID NO: 66,
(b) the CDR-H2 comprising the amino acid sequence of SEQ ID NO: 98, and
(c) the CDR-H3 comprising the amino acid sequence of SEQ ID NO: 130;
or
(a) the CDR-H1 comprising the amino acid sequence of SEQ ID NO: 67,
(b) the CDR-H2 comprising the amino acid sequence of SEQ ID NO: 99, and
(c) the CDR-H3 comprising the amino acid sequence of SEQ ID NO: 131;
or
(a) the CDR-H1 comprising the amino acid sequence of SEQ ID NO: 68,
(b) the CDR-H2 comprising the amino acid sequence of SEQ ID NO: 100, and
(c) the CDR-H3 comprising the amino acid sequence of SEQ ID NO: 132;
or
(a) the CDR-H1 comprising the amino acid sequence of SEQ ID NO: 69,
(b) the CDR-H2 comprising the amino acid sequence of SEQ ID NO: 101, and
(c) the CDR-H3 comprising the amino acid sequence of SEQ ID NO: 133;
or
(a) the CDR-H1 comprising the amino acid sequence of SEQ ID NO: 70,
(b) the CDR-H2 comprising the amino acid sequence of SEQ ID NO: 102, and
(c) the CDR-H3 comprising the amino acid sequence of SEQ ID NO: 134;
or
(a) the CDR-H1 comprising the amino acid sequence of SEQ ID NO: 71,
(b) the CDR-H2 comprising the amino acid sequence of SEQ ID NO: 103, and
(c) the CDR-H3 comprising the amino acid sequence of SEQ ID NO: 135;
or
(a) the CDR-H1 comprising the amino acid sequence of SEQ ID NO: 72,
(b) the CDR-H2 comprising the amino acid sequence of SEQ ID NO: 104, and
(c) the CDR-H3 comprising the amino acid sequence of SEQ ID NO: 136;
or
(a) the CDR-H1 comprising the amino acid sequence of SEQ ID NO: 73,
(b) the CDR-H2 comprising the amino acid sequence of SEQ ID NO: 105, and
(c) the CDR-H3 comprising the amino acid sequence of SEQ ID NO: 137;
or
(a) the CDR-H1 comprising the amino acid sequence of SEQ ID NO: 74,
(b) the CDR-H2 comprising the amino acid sequence of SEQ ID NO: 106, and
(c) the CDR-H3 comprising the amino acid sequence of SEQ ID NO: 138;
or
(a) the CDR-H1 comprising the amino acid sequence of SEQ ID NO: 75,
(b) the CDR-H2 comprising the amino acid sequence of SEQ ID NO: 107, and
(c) the CDR-H3 comprising the amino acid sequence of SEQ ID NO: 139.

26.-41. (canceled)

42. The antibody fragment of claim 1, comprising:
(a) the CDR-L1 comprising the amino acid sequence selected from the group consisting of SEQ ID NOs: 141-150 or a variant thereof having a single substitution, deletion or insertion from any one of SEQ ID NOs: 141-150;
(b) the CDR-L2 comprising an amino acid sequence selected from the group consisting of SEQ ID NOs: 152-161 or a variant thereof having a single substitution, deletion or insertion from any one of SEQ ID NOs: 152-161; and
(c) the CDR-L3 comprising an amino acid sequence selected from the group consisting of SEQ ID NOs: 163-172 or a variant thereof having a single substitution, deletion or insertion from any one of SEQ ID NOs: 163-172.

43. The antibody fragment of claim 1, comprising:
(a) the CDR-L1 comprising the amino acid sequence of SEQ ID NO: 14,
(b) the CDR-L2 comprising the amino acid sequence of SEQ ID NO: 152, and
(c) the CDR-L3 comprising the amino acid sequence of SEQ ID NO: 163;
or
(a) the CDR-L1 comprising the amino acid sequence of SEQ ID NO: 142,
(b) the CDR-L2 comprising the amino acid sequence of SEQ ID NO: 153, and
(c) the CDR-L3 comprising the amino acid sequence of SEQ ID NO: 164;
or
(a) the CDR-L1 comprising the amino acid sequence of SEQ ID NO: 143,
(b) the CDR-L2 comprising the amino acid sequence of SEQ ID NO: 154, and
(c) the CDR-L3 comprising the amino acid sequence of SEQ ID NO: 165;
or
(a) the CDR-L1 comprising the amino acid sequence of SEQ ID NO: 144,
(b) the CDR-L2 comprising the amino acid sequence of SEQ ID NO: 155, and
(c) the CDR-L3 comprising the amino acid sequence of SEQ ID NO: 166;
or
(a) the CDR-L1 comprising the amino acid sequence of SEQ ID NO: 145,
(b) the CDR-L2 comprising the amino acid sequence of SEQ ID NO: 156, and
(c) the CDR-L3 comprising the amino acid sequence of SEQ ID NO: 167;
or
(a) the CDR-L1 comprising the amino acid sequence of SEQ ID NO: 146,
(b) the CDR-L2 comprising the amino acid sequence of SEQ ID NO: 157, and
(c) the CDR-L3 comprising the amino acid sequence of SEQ ID NO: 168;
or
(a) the CDR-L1 comprising the amino acid sequence of SEQ ID NO: 147,
(b) the CDR-L2 comprising the amino acid sequence of SEQ ID NO: 158, and
(c) the CDR-L3 comprising the amino acid sequence of SEQ ID NO: 169;
or
(a) the CDR-L1 comprising the amino acid sequence of SEQ ID NO: 148,
(b) the CDR-L2 comprising the amino acid sequence of SEQ ID NO: 159, and
(c) the CDR-L3 comprising the amino acid sequence of SEQ ID NO: 170;
or
(a) the CDR-L1 comprising the amino acid sequence of SEQ ID NO: 149,
(b) the CDR-L2 comprising the amino acid sequence of SEQ ID NO: 160, and
(c) the CDR-L3 comprising the amino acid sequence of SEQ ID NO: 171;
or
(a) the CDR-L1 comprising the amino acid sequence of SEQ ID NO: 150,
(b) the CDR-L2 comprising the amino acid sequence of SEQ ID NO: 161, and
(c) the CDR-L3 comprising the amino acid sequence of SEQ ID NO: 172.

44.-57. (canceled)

58. The antibody or the antibody fragment of claim 21, wherein the antibody or the antibody fragment is an scFab antibody or a scFab fragment and comprises a scFab linker, and wherein the scFab linker comprises an amino acid sequence adapted to present the CDR-H1, CDR-H2, CDR-H3, CDR-L1, CDR-L2, CDR-L3 for binding to at least one sarbecovirus spike protein RBD.

59. (canceled)

60. The antibody or the antibody fragment of claim 58, comprising an amino acid sequence selected from the group consisting of SEQ ID NOs: 186-196, or a variant thereof having a single substitution, deletion, or insertion from SEQ ID NOs: 186-196.

61. The antibody fragment of claim 1, comprising:
a heavy chain amino acid sequence selected from the group consisting of SEQ ID NOs: 1-16, 18-33, and 174-184 or a variant thereof having a single substitution, deletion or insertion from any one of SEQ ID NOs: 1-16, 18-33, and 174-184,
or
a light chain amino acid sequence selected from the group consisting of SEQ ID NOs: 17, 34-43, and 173 or a variant thereof having a single substitution, deletion or insertion from any one of SEQ ID NOs: 17, 34-43, and 173.

62. (canceled)

63. The antibody or fragment thereof of claim 21, wherein the antibody or fragment thereof specifically binds to at least one sarbecovirus spike protein.

64. (canceled)

65. (canceled)

66. The antibody or fragment thereof of claim 1, wherein the antibody or fragment thereof inhibits infectivity of two or more different sarbecoviruses.

67.-70. (canceled)

71. The antibody or fragment thereof of claim 21, wherein the sarbecovirus is SARS-CoV-2 or a variant thereof, SARS-CoV or a variant thereof, WIV1 or a variant thereof, SHC014 or a variant thereof, BtKY72 or a variant thereof, Khosta2/SARS-CoV Chimera or a variant thereof, or LyRa3/SARS-CoV Chimera or a variant thereof; and optionally the SARS-CoV-2 variant is Wuhan (WA1 D614G), Beta, B.1.1.7 (20I/501Y.V1), B.1.351 (20H/501Y.V2), P.1 (20J/501Y.V3), Delta, Omicron BA.1, Omicron BA.2, Omicron BA.4, or Omicron BA.5.

72. The antibody or fragment thereof of claim 21, wherein the antibody or fragment thereof is capable of binding to each of the three RBDs of a sarbecovirus spike trimer.

73. A composition comprising the antibody or fragment thereof of claim 1 and a pharmaceutically acceptable carrier.

74. A polynucleotide encoding one or more of the antibody or fragment thereof of claim 1.

75. (canceled)

76. (canceled)

77. A method of treating or preventing a coronavirus infection in a patient in need thereof, comprising administering to the patient an effective amount of the antibody or fragment thereof of claim 1 or a composition thereof.

78. The method of claim 77, wherein the coronavirus is a coronavirus in the genus of Alpha-coronavirus, Beta-coronavirus, Delta-coronavirus, or Omicron-coronavirus;
and optionally wherein the coronavirus is a coronavirus of the subgenus Sarbecovirus.

79. The method of claim 77, wherein the coronavirus is SARS-CoV-2 and variants thereof, B.1.1.7 (20I/501Y.V1), B.1.351 (20H/501Y.V2), P.1 (20J/501Y.V3), RsSTT200 and variants thereof, Pang17 and variants thereof, RaTG13 and variants thereof, SARS-CoV and variants thereof, WIV1 and variants thereof, SHC014 and variants thereof, LyRa3 and variants thereof, C028 and variants thereof, Rs4081 and variants thereof, RmYN02 and variants thereof, Rf1 and variants thereof, Yun11 and variants thereof, BM4831 and variants thereof, BtKY72 and variants thereof, or Khosta2 and variants thereof.

80. The method of claim 77, further comprising administering to the patient a second therapeutic agent; optionally the second therapeutic agent comprises an anti-viral compound, an immunosuppressant, an antibody, or any combination thereof; and further optionally wherein the second therapeutic agent comprises remdesivir, molnupiravir, tocilizumab, favipiravir, merimepodib, artesunate, favipiravir, ribavirin, EIDD-2801, niclosamide, nitazoxanide, oseltamivir, AT-527, paxlovid, regdanvimab, ramdicivir, baricitinib, imatinib, casirivimab, imdevimab, bemcentinib, bamlanivimab, etesevimab, sotrovimab, leronlimab, bebtelovimab, cilgavimab, IMU-838, oseltamivir, or dexamethasone.


SEQUENCE LISTING (196 sequences)

SEQ ID NO: 1 - protein, 454 aa, synthetic construct
EVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRGDDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 454 aa, synthetic construct
EVQLVESGGGVVQPGRSLRLSCAASGFPFSIYGMHWVRQAPGKGLEWVAVISYDGSNKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVGVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 454 aa, synthetic construct
EVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPSDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 454 aa, synthetic construct
EVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRSSDIVLVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIWHVSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 454 aa, synthetic construct
EVQLVESGGGVVQPGRSLRLSCAASGFDYPISGMHWVRQAPGKGLEWVAHISYDGSYKYYADSVKGRFTISRDNSKNTLYLQMNSLRAEDTAVYYCAKEGRPSDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 454 aa, synthetic construct
EVQLVESGGGVVQPGRSLRLSCAASGFDFPIAGMHWVRQAPGKGLEWVAHISYDGSHKYYADSVKGRFTISRDNSKNTLYLQMNSLRAEDTAVYYCAKEGRPSDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 454 aa, synthetic construct
EVQLVESGGGVVQPGRSLRLSCAASGFDYPIYGMHWVRQAPGKGLEWVAVISYDGSNKYYADSVKGRFTISRDNSKNTLYLQMNSLRAEDTAVYYCAKEGRPSDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 454 aa, synthetic construct
EVQLVESGGGVVQPGRSLRLSCAASGFPFSIYGMHWVRQAPGKGLEWVAVISYDGSNKYYADSVKGRFTISRDNSKNTLYLQMNSLRAEDTAVYYCAKEGRPSDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 454 aa, synthetic construct
EVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSHSLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 457 aa, synthetic construct
QVQLVESGGGVVQPGRSLRLSCAASGFEFSNYAMHWVRQAPGKGLEWVAVINYDGSNKYYADSVKGRFTISRDNSKNTLYLQMNSLRAEDTAIYYCASGYTGYDYFFHGEYFGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 457 aa, synthetic construct
QVQLVESGGGVVQPGRSLRLSCAASGFHFSHYAMHWVRQAPGKGLEWVAVINYDGSNKYYADSVKGRFTISRDNSKNTLYLQMNSLRAEDTAIYYCASGYTGYDYFFGGEYFGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 457 aa, synthetic construct
QVQLVESGGGVVQPGRSLRLSCAASGFAFYNYAMHWVRQAPGKGLEWVAVINYDGSNKYYADSVKGRFTISRDNSKNTLYLQMNSLRAEDTAIYYCASGYTGYDYFFGGEYFGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 457 aa, synthetic construct; identical to SEQ ID NO: 20

SEQ ID NO: 22 - protein, 457 aa, synthetic construct
QVQLVESGGGVVQPGRSLRLSCAASGFKFSHYAMHWVRQAPGKGLEWVAVINFDGSNKYYADSVKGRFTISRDNSKNTLYLQMNSLRAEDTAIYYCASGYTGYDYFFNGEYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 457 aa, synthetic construct
QVQLVESGGGVVQPGRSLRLSCAASGFHFNNYAMHWVRQAPGKGLEWVAVISYDGTNKYYADSVKGRFTISRDNSKNTLYLQMNSLRAEDTAIYYCASGYTGYDYFFGGDYFGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 457 aa, synthetic construct
QVQLVESGGGVVQPGRSLRLSCAASGFTFNNYAMHWVRQAPGKGLEWVAVISYDGSNKYYADSVKGRFTISRDNSKNTLYLQMNSLRAEDTAIYYCASGYTGYDYFFGGDYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 457 aa, synthetic construct
QVQLVESGGGVVQPGRSLRLSCAASGFKFSNYAMHWVRQAPGKGLEWVAVISYDGSNKYYADSVKGRFTISRDNSKNTLYLQMNSLRAEDTAIYYCASGYTGYDYFFRGEYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 457 aa, synthetic construct
QVQLVESGGGVVQPGRSLRLSCAASGFHFSNYAMHWVRQAPGKGLEWVAVINFDGSNKYYADSVKGRFTISRDNSKNTLYLQMNSLRAEDTAIYYCASGYTGYDYFFSGEYFGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 457 aa, synthetic construct
QVQLVESGGGVVQPGRSLRLSCAASGFHFNHYAMHWVRQAPGKGLEWVAVINYDGTNKYYADSVKGRFTISRDNSKNTLYLQMNSLRAEDTAIYYCASGYTGYDYFFHGEYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 457 aa, synthetic construct
QVQLVESGGGVVQPGRSLRLSCAASGFAFYNYAMHWVRQAPGKGLEWVAVINYDGSNKYYADSVKGRFTISRDNSKNTLYLQMNSLRAEDTAIYYCASGYTGYDYFVGGEYFGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 457 aa, synthetic construct
QVQLVESGGGVVQPGRSLRLSCAASGFTFSNYAMHWVRQAPGKGLEWVAVINFDGSNKYYADSVKGRFTISRDNSKNTLYLQMNSLRAEDTAIYYCASGYTGYDYFVNGEYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 457 aa, synthetic construct
QVQLVESGGGVVQPGRSLRLSCAASGFAFYNYAMHWVRQAPGKGLEWVAVINYDGTNKYYADSVKGRFTISRDNSKNTLYLQMNSLRAEDTAIYYCASGYTGYDYFFGGEYYGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 457 aa, synthetic construct
QVQLVESGGGVVQPGRSLRLSCAASGFHFSHYAMHWVRQAPGKGLEWVAVISFDGSNKYYADSVKGRFTISRDNSKNTLYLQMNSLRAEDTAIYYCASGYTGYDYFFNGEYFGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 457 aa, synthetic construct
QVQLVESGGGVVQPGRSLRLSCAASGFYFRNYAMHWVRQAPGKGLEWVAVINYDGSNKYYADSVKGRFTISRDNSKNTLYLQMNSLRAEDTAIYYCASGYTGYDYFVGGEYFGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 457 aa, synthetic construct
QVQLVESGGGVVQPGRSLRLSCAASGFYFKNYAMHWVRQAPGKGLEWVAVINYDGSNKYYADSVKGRFTISRDNSKNTLYLQMNSLRAEDTAIYYCASGYTGYDYFVGGEYFGLEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 217 aa, synthetic construct
QPVLTQSPSASASLGASVKLTCTLSSGHSSYAIAWHQQQPEKGPRYLMKLNTDGSFNKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 217 aa, synthetic construct
QPVLTQSPSASASLGASVKLTCTLSSGHSSYAIAWHQQQPEKGPRYLMKLNTDGSLEKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 217 aa, synthetic construct
QPVLTQSPSASASLGASVKLTCTLSSGHSSYAIAWHQQQPEKGPRYLMKLNTDGSYEKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 217 aa, synthetic construct
QPVLTQSPSASASLGASVKLTCTLSSGHSSYAIAWHQQQPEKGPRYLMKLNTDGSYDKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 217 aa, synthetic construct
QPVLTQSPSASASLGASVKLTCTLSSGHSSYAIAWHQQQPEKGPRYLMKLNTDGSFEKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 217 aa, synthetic construct
QPVLTQSPSASASLGASVKLTCTLSSGHSSYAIAWHQQQPEKGPRYLMKLNTDGSLDKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 217 aa, synthetic construct
QPVLTQSPSASASLGASVKLTCTLSSGHSSYAIAWHQQQPEKGPRYLMKLNTDGSFDKGDGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 217 aa, synthetic construct
QPVLTQSPSASASLGASVKLTCTLSSGHSSYAIAWHQQQPEKGPRYLMKLATDGSYEMGHGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 217 aa, synthetic construct
QPVLTQSPSASASLGASVKLTCTLSSGHSSYAIAWHQQQPEKGPRYLMKLNTDGSYEMGVGIPDRFSGSSSGAERYLTISSLQSEDEADYYCQTWGTGILVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 217 aa, synthetic construct; identical to SEQ ID NO: 38

SEQ ID NO: 44 - protein, 5 aa, synthetic construct
IFGMH

SEQ ID NO: 45 - protein, 5 aa, synthetic construct
IYGMH

SEQ ID NO: 46 - protein, 5 aa, synthetic construct
IYGMH

SEQ ID NO: 47 - protein, 5 aa, synthetic construct
IYGMH

SEQ ID NO: 48 - protein, 5 aa, synthetic construct
IYGMH

SEQ ID NO: 49 - protein, 5 aa, synthetic construct
IFGMH

SEQ ID NO: 50 - protein, 5 aa, synthetic construct
IFGMH

SEQ ID NO: 51 - protein, 5 aa, synthetic construct
IYGMH

SEQ ID NO: 52 - protein, 5 aa, synthetic construct
IYGMH

SEQ ID NO: 53 - protein, 5 aa, synthetic construct
IYGMH

SEQ ID NO: 54 - protein, 5 aa, synthetic construct
IYGMH

SEQ ID NO: 55 - protein, 5 aa, synthetic construct
ISGMH

SEQ ID NO: 56 - protein, 5 aa, synthetic construct
IAGMH

SEQ ID NO: 57 - protein, 5 aa, synthetic construct
IYGMH

SEQ ID NO: 58 - protein, 5 aa, synthetic construct
IYGMH

SEQ ID NO: 59 - protein, 5 aa, synthetic construct
IYGMH

SEQ ID NO: 60 - protein, 5 aa, synthetic construct
NYAMH

SEQ ID NO: 61 - protein, 5 aa, synthetic construct
HYAMH

SEQ ID NO: 62 - protein, 5 aa, synthetic construct
NYAMH

SEQ ID NO: 63 - protein, 5 aa, synthetic construct
NYAMH

SEQ ID NO: 64 - protein, 5 aa, synthetic construct
HYAMH

SEQ ID NO: 65 - protein, 5 aa, synthetic construct
NYAMH

SEQ ID NO: 66 - protein, 5 aa, synthetic construct
NYAMH

SEQ ID NO: 67 - protein, 5 aa, synthetic construct
NYAMH

SEQ ID NO: 68 - protein, 5 aa, synthetic construct
NYAMH

SEQ ID NO: 69 - protein, 5 aa, synthetic construct
HYAMH

SEQ ID NO: 70 - protein, 5 aa, synthetic construct
NYAMH

SEQ ID NO: 71 - protein, 5 aa, synthetic construct
NYAMH

SEQ ID NO: 72 - protein, 5 aa, synthetic construct
NYAMH

SEQ ID NO: 73 - protein, 5 aa, synthetic construct
HYAMH

SEQ ID NO: 74 - protein, 5 aa, synthetic construct
NYAMH

SEQ ID NO: 75 - protein, 5 aa, synthetic construct
NYAMH

SEQ ID NO: 76 - protein, 17 aa, synthetic construct
VISYDGSHKYYADSVKG

SEQ ID NO: 77 - protein, 17 aa, synthetic construct
VISYDGSHKYYADSVKG

SEQ ID NO: 78 - protein, 17 aa, synthetic construct
VISYDGSNKYYADSVKG

SEQ ID NO: 79 - protein, 17 aa, synthetic construct
VISYDGSHKYYADSVKG

SEQ ID NO: 80 - protein, 17 aa, synthetic construct
VISYDGSHKYYADSVKG

SEQ ID NO: 81 - protein, 17 aa, synthetic construct
VISYDGSHKYYADSVKG

SEQ ID NO: 82 - protein, 17 aa, synthetic construct
VISYDGSHKYYADSVKG

SEQ ID NO: 83 - protein, 17 aa, synthetic construct
VISYDGSHKYYADSVKG

SEQ ID NO: 84 - protein, 17 aa, synthetic construct
VISYDGSHKYYADSVKG

SEQ ID NO: 85 - protein, 17 aa, synthetic construct
VISYDGSHKYYADSVKG

SEQ ID NO: 86 - protein, 17 aa, synthetic construct
VISYDGSHKYYADSVKG

SEQ ID NO: 87 - protein, 17 aa, synthetic construct
HISYDGSYKYYADSVKG

SEQ ID NO: 88 - protein, 17 aa, synthetic construct
HISYDGSHKYYADSVKG

SEQ ID NO: 89 - protein, 17 aa, synthetic construct
VISYDGSNKYYADSVKG

SEQ ID NO: 90 - protein, 17 aa, synthetic construct
VISYDGSNKYYADSVKG

SEQ ID NO: 91 - protein, 17 aa, synthetic construct
VISYDGSHKYYADSVKG

SEQ ID NO: 92 - protein, 17 aa, synthetic construct
VINYDGSNKYYADSVKG

SEQ ID NO: 93 - protein, 17 aa, synthetic construct
VINYDGSNKYYADSVKG

SEQ ID NO: 94 - protein, 17 aa, synthetic construct
VINYDGSNKYYADSVKG

SEQ ID NO: 95 - protein, 17 aa, synthetic construct
VINFDGSNKYYADSVKG

SEQ ID NO: 96 - protein, 17 aa, synthetic construct
VINFDGSNKYYADSVKG

SEQ ID NO: 97 - protein, 17 aa, synthetic construct
VISYDGTNKYYADSVKG

SEQ ID NO: 98 - protein, 17 aa, synthetic construct
VISYDGSNKYYADSVKG

SEQ ID NO: 99 - protein, 17 aa, synthetic construct
VISYDGSNKYYADSVKG

SEQ ID NO: 100 - protein, 17 aa, synthetic construct
VINFDGSNKYYADSVKG

SEQ ID NO: 101 - protein, 17 aa, synthetic construct
VINYDGTNKYYADSVKG

SEQ ID NO: 102 - protein, 17 aa, synthetic construct
VINYDGSNKYYADSVKG

SEQ ID NO: 103 - protein, 17 aa, synthetic construct
VINFDGSNKYYADSVKG

SEQ ID NO: 104 - protein, 17 aa, synthetic construct
VINYDGTNKYYADSVKG

SEQ ID NO: 105 - protein, 17 aa, synthetic construct
VISFDGSNKYYADSVKG

SEQ ID NO: 106 - protein, 17 aa, synthetic construct
VINYDGSNKYYADSVKG

SEQ ID NO: 107 - protein, 17 aa, synthetic construct
VINYDGSNKYYADSVKG

SEQ ID NO: 108 - protein, 15 aa, synthetic construct
EGRPDDIVHVVAFDY

SEQ ID NO: 109 - protein, 15 aa, synthetic construct
EGRGDDIVVVVAFDY

SEQ ID NO: 110 - protein, 15 aa, synthetic construct
EGRPDDIVRVVAFDY

SEQ ID NO: 111 - protein, 15 aa, synthetic construct
EGRPSDIVHVVAFDY

SEQ ID NO: 112 - protein, 15 aa, synthetic construct
EGRPSDIVGVVAFDY

SEQ ID NO: 113 - protein, 15 aa, synthetic construct
EGRPSDIVHVVAFDY

SEQ ID NO: 114 - protein, 15 aa, synthetic construct
EGRPDDIVRVVAFDY

SEQ ID NO: 115 - protein, 15 aa, synthetic construct
EGRSSDIVLVVAFDY

SEQ ID NO: 116 - protein, 15 aa, synthetic construct
EGRPSDIWHVSAFDY

SEQ ID NO: 117 - protein, 15 aa, synthetic construct
EGRPDDIVHVVAFDY

SEQ ID NO: 118 - protein, 15 aa, synthetic construct
EGRPDDIVRVVAFDY

SEQ ID NO: 119 - protein, 15 aa, synthetic construct
EGRPSDIVVVVAFDY

SEQ ID NO: 120 - protein, 15 aa, synthetic construct
EGRPSDIVVVVAFDY

SEQ ID NO: 121 - protein, 15 aa, synthetic construct
EGRPSDIVVVVAFDY

SEQ ID NO: 122 - protein, 15 aa, synthetic construct
EGRPSDIVVVVAFDY

SEQ ID NO: 123 - protein, 15 aa, synthetic construct
EGRPSDIVVVVAFDY

SEQ ID NO: 124 - protein, 18 aa, synthetic construct
GYTGYDYFFHGEYFGLEV

SEQ ID NO: 125 - protein, 18 aa, synthetic construct
GYTGYDYFFGGEYFGLDV

SEQ ID NO: 126 - protein, 18 aa, synthetic construct
GYTGYDYFFGGEYFGLDV

SEQ ID NO: 127 - protein, 18 aa, synthetic construct
GYTGYDYFFGGDYFGLEV

SEQ ID NO: 128 - protein, 18 aa, synthetic construct
GYTGYDYFFNGEYYGLDV

SEQ ID NO: 129 - protein, 18 aa, synthetic construct
GYTGYDYFFGGDYFGLDV

SEQ ID NO: 130 - protein, 18 aa, synthetic construct
GYTGYDYFFGGDYYGLDV

SEQ ID NO: 131 - protein, 18 aa, synthetic construct
GYTGYDYFFRGEYYGLDV

SEQ ID NO: 132 - protein, 18 aa, synthetic construct
GYTGYDYFFSGEYFGLEV

SEQ ID NO: 133 - protein, 18 aa, synthetic construct
GYTGYDYFFHGEYYGLDV

SEQ ID NO: 134 - protein, 18 aa, synthetic construct
GYTGYDYFVGGEYFGLEV

SEQ ID NO: 135 - protein, 18 aa, synthetic construct
GYTGYDYFVNGEYYGLDV

SEQ ID NO: 136 - protein, 18 aa, synthetic construct
GYTGYDYFFGGEYYGLEV

SEQ ID NO: 137 - protein, 18 aa, synthetic construct
GYTGYDYFFNGEYFGLDV

SEQ ID NO: 138 - protein, 18 aa, synthetic construct
GYTGYDYFVGGEYFGLEV

SEQ ID NO: 139 - protein, 18 aa, synthetic construct
GYTGYDYFVGGEYFGLEV

SEQ ID NO: 140 - protein, 11 aa, synthetic construct
CRASQSISSYL

SEQ ID NO: 141 - protein, 12 aa, synthetic construct
TLSSGHSSYAIA

SEQ ID NO: 142 - protein, 12 aa, synthetic construct
TLSSGHSSYAIA

SEQ ID NO: 143 - protein, 12 aa, synthetic construct
TLSSGHSSYAIA

SEQ ID NO: 144 - protein, 12 aa, synthetic construct
TLSSGHSSYAIA

SEQ ID NO: 145 - protein, 12 aa, synthetic construct
TLSSGHSSYAIA

SEQ ID NO: 146 - protein, 12 aa, synthetic construct
TLSSGHSSYAIA

SEQ ID NO: 147 - protein, 12 aa, synthetic construct
TLSSGHSSYAIA

SEQ ID NO: 148 - protein, 12 aa, synthetic construct
TLSSGHSSYAIA

SEQ ID NO: 149 - protein, 12 aa, synthetic construct
TLSSGHSSYAIA

SEQ ID NO: 150 - protein, 12 aa, synthetic construct
TLSSGHSSYAIA

SEQ ID NO: 151 - protein, 7 aa, synthetic construct
YAASSLQ

SEQ ID NO: 152 - protein, 11 aa, synthetic construct
LNTDGSFNKGD

SEQ ID NO: 153 - protein, 11 aa, synthetic construct
LNTDGSLEKGD

SEQ ID NO: 154 - protein, 11 aa, synthetic construct
LNTDGSYEKGD

SEQ ID NO: 155 - protein, 11 aa, synthetic construct
LNTDGSYDKGD

SEQ ID NO: 156 - protein, 11 aa, synthetic construct
LNTDGSFEKGD

SEQ ID NO: 157 - protein, 11 aa, synthetic construct
LNTDGSLDKGD

SEQ ID NO: 158 - protein, 11 aa, synthetic construct
LNTDGSFDKGD

SEQ ID NO: 159 - protein, 11 aa, synthetic construct
LATDGSYEMGH

SEQ ID NO: 160 - protein, 11 aa, synthetic construct
LNTDGSYEMGV

SEQ ID NO: 161 - protein, 11 aa, synthetic construct
LNTDGSFEKGD

SEQ ID NO: 162 - protein, 9 aa, synthetic construct
CQQSHSLPR

SEQ ID NO: 163 - protein, 9 aa, synthetic construct
QTWGTGILV

SEQ ID NO: 164 - protein, 9 aa, synthetic construct
QTWGTGILV

SEQ ID NO: 165 - protein, 9 aa, synthetic construct
QTWGTGILV

SEQ ID NO: 166 - protein, 9 aa, synthetic construct
QTWGTGILV

SEQ ID NO: 167 - protein, 9 aa, synthetic construct
QTWGTGILV

SEQ ID NO: 168 - protein, 9 aa, synthetic construct
QTWGTGILV

SEQ ID NO: 169 - protein, 9 aa, synthetic construct
QTWGTGILV

SEQ ID NO: 170 - protein, 9 aa, synthetic construct
QTWGTGILV

SEQ ID NO: 171 - protein, 9 aa, synthetic construct
QTWGTGILV

SEQ ID NO: 172 - protein, 9 aa, synthetic construct
QTWGTGILV

SEQ ID NO: 173 - protein, 233 aa, synthetic construct
MGWSCIILFLVATATGVHSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 473 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRGDDIVVVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 473 aa, synthetic construct
MGWSCIILFLVATATGVHSEVQLVESGGGVVQPGRSLRLSCAASGFPFSIYGMHWVRQAPGKGLEWVAVISYDGSNKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVGVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 473 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPSDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 473 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRSSDIVLVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIWHVSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVHVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 473 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVRVVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 15 aa, synthetic construct
SGGGGSGGGGSGGGG

SEQ ID NO: 186 - protein, 265 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVHVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 187 - protein, 265 aa, synthetic construct; identical to SEQ ID NO: 186

SEQ ID NO: 188 - protein, 265 aa, synthetic construct
MGWSCIILFLVATATGVHSEVQLVESGGGVVQPGRSLRLSCAASGFPFSIYGMHWVRQAPGKGLEWVAVISYDGSNKYYADSVKGRFTISRDNSKNTLYLQMNSLRAEDTAVYYCAKEGRPDDIVRVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 189 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVHVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 190 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIVGVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 191 - protein, 265 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPSDIVHVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 192 - protein, 265 aa, synthetic construct
MGWSCIILFLVATATGVHSEVQLVESGGGVVQPGRSLRLSCAASGFPFSIFGMHWVRQAPGKGLEWVAVISYDGSHKYYADSVKGRFTISRDNSKNTLYLQMNSLRAEDTAVYYCAKEGRPDDIVRVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 193 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRSSDIVLVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 194 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPSDIWHVSAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 195 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVHVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 196 - protein, 265 aa, synthetic construct
MGWSCIILFLVATATGVHSEVQLVESGGGVVQPGRSLRLSCAASGFPFSIYGMHWVRQAPGKGLEWVAVISYDGSHKYYADSVKGRFTISRDNSKNTLYLQMNSLRAEDTAVYYCAKEGRPDDIVRVVAFDYWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK